(19) United States
(12) Patent Application Publication
(10) Pub. No.: US 2003/0211605 A1
(43) Pub. Date: Nov. 13, 2003

(54) Derivation of midbrain dopaminergic neurons from embryonic stem cells

(76) Inventors: Sang-Hun Lee, Seoul (KR); Nadya Lumelsky, Washington, DC (US); Lorenz Studer, New York, NY (US); Ronald D.G. McKay, Bethesda, MD (US); Jonathan Auerbach, Bethesda, MD (US); Jong-Hoon Kim, Rockville, MD (US)

Correspondence Address: KLARQUIST SPARKMAN, LLP, 121 SW SALMON STREET, SUITE 1600, PORTLAND, OR, 97204, US

(21) Appl. No.: 10/258,975

(22) PCT Filed: May 1, 2001
(86) PCT No.: PCT/US01/14051

(51) Int. Cl. C12N 5/08

(52) U.S. Cl. 435/368

(57) ABSTRACT

The invention provides a method of culturing cells. The method generally includes live stages: (1) expansion of ES cells; (2) generation of embryoid bodies; (3) selection of CNS precursor cells; (4) expansion of CNS precursor cells; and (5) differentiation of CNS precursor cells. During the expansion phase, the CNS precursor cells are cultured in a media which includes at least one neurologic agent such as bFGF, SHH, and FGF-8. The expanded CNS precursors are differentiated by withdrawal of at least one neurologic agent, typically, bFGF. Preferably, the differentiation media includes ascorbic acid. The method of the invention can be used to culture a variety of cells, preferably neuronal cells, including, but not limited to dopaminergic neuron cells, cholinergic neuronal cells and serotonergic neuron cells. The invention also provides a method for treating a neurological disorder, such as Parkinson's disease, a method of introducing a gene product into a brain of a patient, and an assay for neurologically active substances. The invention further provides a cell culture which includes differentiated neuron cells, of which at least about 20 % of the differentiated neurons are dopaminergic neurons.

This application is being filed as a PCT International Patent application in the name of Sang-Hun Lee, Nadya Lumelsky, Lorenz Studer, and Ron McKay, applicants for all countries, on May 1, 2001.

### BACKGROUND OF THE INVENTION

Parkinson's disease is a neurodegenerative disorder where midbrain dopaminergic neurons are specifically destroyed. It is characterized by gradual, progressive muscle rigidity, tremors and clumsiness and affects an estimated one million patients in the United States. Although Parkinson's disease may be ascribed to the use of some medications, such as phenothiazine tranquilizers; brain injury; tumors; post-influenza encephalitis; slow-virus infection; carbon-monoxide poisoning; or agricultural chemicals, the cause of Parkinson's disease is generally unknown.

Parkinson's disease is currently considered incurable. However, prescription medications for symptomatic relief of Parkinson's disease are available and include anticholinergics; antihistamines; antitremor drugs, such as amantadine; or antiparkinson medications, including bromocriptine, levodopa and carbidopa. Although these drugs may decrease tremors and reduce muscle rigidity, they often have significant side effects. Additionally, these drugs only relieve the symptoms and do not cure the disease.

Thus, several strategies are being pursued to develop new therapies for Parkinsonian patients. These techniques range from the use of dopaminotrophic factors (Takayama et al. (1995) “Basic fibroblast growth factor increases dopaminergic graft survival and function in a rat model of Parkinson's disease,” Nature Med. 1:53-58) and viral vectors (Choi-Lundberg et al., (1997) “Dopaminergic neurons protected from degeneration by GDNF gene therapy,” Science 275:838-841) to the transplantation of primary xenogenic tissue (Deacon et al, (1997) “Histological evidence of fetal pig neural cell survival after transplantation into a patient with Parkinson's disease,” Nature Med. 3:350-353).

Transplantation of dopaminergic neurons is a clinically promising experimental treatment in late stage Parkinson's disease. More than 200 patients have been transplanted worldwide (Olanow et al., (1996) “Fetal nigral transplantation as a therapy for Parkinson's disease,” Trends Neurosci. 19:102-109). Clinical improvement has been confirmed (Olanow et al, supra and Wenning et al., (1997) “Short- and long-term survival and function of unilateral intrastriatal dopaminergic grafts in Parkinson's disease,” Ann. Neurol. 42:95-107) and was correlated to good graft survival and innervation of the host striatum (Kordower et al., (1995) “Neuropathological evidence of graft survival and striatal reinnervation after the transplantation of fetal mesencephalic tissue in a patient with Parkinson's disease,” N. Engl. J. Med. 332:1118-1124). However, fetal nigral transplantation therapy generally requires human fetal tissue from at least 3-5 embryos to obtain a clinically reliable improvement in the patient. This poses an enormous logistical and ethical dilemma.

To address this logistical and ethical dilemma, alternative sources for dopaminergic neurons are being investigated. For example, dopaminergic neurons have been generated from CNS precursor cells (PCT Application “Cell Expansion System for use in Neural Transplantation” Serial No. PCT/US99/16825; and Studer et al., (1998) “Transplantation of expanded mesencephalic precursors leads to recovery in Parkinsonian rats,” Nature Neurosci. 1:290-295.). These precursor-derived neurons are functional in vitro and in vivo and restore behavioral deficits in a rat model of Parkinson's disease.

Even though the primary mesencephalic CNS stem cell culture can provide differentiated dopaminergic neurons suitable for use in cell therapy for Parkinson's disorder, the cell number provided by this method is limited. The percentage of differentiated dopaminergic neurons obtained from expanded mesencephalic precursors decreases as the cells are expanded more than about 10-100 fold. While mesencephalic precursors can generate about 10% to 15% dopaminergic neurons (out of total cell number) after 10-100 fold expansion, when the precursors are expanded 1000 fold, that number drops to only about 1%.

In contrast to mesencephalic precursor cells, ES cells can proliferate indefinitely in an undifferentiated state. Furthermore, embryonic stem (ES) cells are totipotent cells, meaning that they can generate all of the cells present in the body (bone, muscle, brain cells, etc.).

ES cells have been isolated from the inner cell mass of the developing murine blastocyst (Evans et al., (1981), “Establishment in culture of pluripotential cells from mouse embryos,” Nature 292:154-156; Martin et al., (1981) “Isolation of a pluripotent cell line from early mouse embryos cultured in medium conditioned by teracarcinoma stem cells,” Proc. Natl. Acad. Sci. 78:7634-7636; Robertson et al., (1986) “Germ line transmission of a gene introduced into cultured pluripotential cells by a retroviral vector,” Nature 323:445-448; Doetschman et al., (1987) “Targeted correction of a mutant HPRT gene in mouse embryonic stem cells,” Nature 330:576-578; and “Thomas et al., (1987) “Site directed mutagenesis by gene targeting in mouse embryo-derived stem cells,” Cell 51:503-512). Additionally, human cells with ES properties have recently been isolated from the inner blastocyst cell mass (Thomson et al., (1998) “Embryonic stem cell lines derived from human blastocysts,” Science 282:1145-1147) and developing germ cells (Shamblott et al., (1998) “Derivation of pluripotent stem cells from cultured human primordal germ cells,” Proc. Natl. Acad. Sci. U.S.A. 95:13726-13731).

ES cells have been shown to differentiate into neurons and glial cells in vitro (Bain et al., (1995) “Embryonic stem cells express neuronal properties in vitro,” Dev. Biol. 168:342-357; and Okabe et al., (1996) “Development of neuronal precursor cells and functional postmitotic neurons from embryonic stem cells in vitro,” Mech. Dev. 59:89-102) and in vivo (13 rustle et al., (1999) “Embryonic stem cell-derived glial precursors: A source of myelinating transplants,” Science 285:754-756; Deacon et al., (1998) “Blastula-stage stem cells can differentiate into dopaminergic and serotonergic neurons after transplantation,” Exp. Neurol. 149:28-41; and Brustle et al., (1997) “In vitro generated neural precursors participate in mammalian brain development,” Proc. Natl. Acad. Sci. 94:14809-14814). These studies indicate that ES cells differentiate into CNS stem cells that subsequently give rise to neurons and glia. Recent work (Brustle et al., (1999) “Embryonic stem cell-derived glial precursors: A source of myelinating transplants,” Science 285:754-756) demonstrates the efficient derivation of a specific glial fate, oligoendrocytes, after selective expansion of ES derived CNS progeny.

Although neurons expressing glutamate, GABA, and glycine derived from ES cells have been reported, no protocol is currently available for the generation of catecholamine neurons, such as dopaminergic neurons. Generation of dopaminergic neurons is of particular interest in view of the therapeutic promise of cell therapy in Parkinson's disease.

### SUMMARY OF THE INVENTION

A first aspect of the invention provides a method of generating midbrain neurons from embryonic stem (ES) cells. The method generally includes five stages: (1) expansion of ES cells; (2) generation of embryoid bodies; (3) culturing embryoid bodies to select for CNS precursor cells; (4) expansion of CNS precursor cells; and (5) differentiation of CNS precursor cells.

In the first stage, expansion of ES cells, the number of ES cells in increased. Generally, this stage includes a step of incubating ES cells in ES growth medium in the presence of LIF (Leukemia Inhibitory Factor) on gelatin-coated tissue culture plate. Preferably, the media is supplemented with fetal calf serum (FCS), non-essential amino acids, 2-mercaptoethanol, L-glutamine, and antibiotics.

In the second stage, embryoid bodies are generated. Generally, this stage includes culturing the expanded ES cells in ES growth medium in the presence of LIF, but in suspension on a culture plate. More preferably, the embryoid bodies are cultured from a population that contains a majority of (e.g., greater than 50%, more preferably greater than 75%) individual ES cells, rather than clusters of ES cells (e.g. aggregations of 2 or more ES cells, typically about 10 or more ES cells).

In the third stage, the embryoid bodies are cultured under conditions to select for CNS precursor cells. Generally, this selection stage includes a step of incubating the embryoid bodies in a medium which selects for CNS precursor cells. Preferably, the medium is a serum free medium supplemented with nutrients such as insulin, selenium chloride, transferrin and fibronectin.

In the fourth stage, the CNS precursor cells are expanded by incubating the cells in CNS proliferation media. A variety of culture media are known and are suitable for use in the invention. Generally, proliferation media includes a minimal essential media such as DMEM and/or F12, preferably supplemented with sodium bicarbonate. Preferably the media does not include 4-(2-hydroxyethyl)-1-piperazine-ethanesulfonic acid (HEPES). A preferred culture media includes N2 supplement. Preferably, the culture media also includes neurologic agents to encourage proliferation of CNS precursor cells. Examples of suitable neurologic agents include basic fibroblast growth factor (bFGF) and epidermal growth factor (EGF). The culture media may also be supplemented with other neurologic agents to increase the efficiency of the generation of midbrain dopaminergic neurons, for example, factors that control dopaminergic and serotonergic cell fates during embryogenesis in vivo. Preferably, the media includes sonic hedgehog (SHH) protein (or functional fragments thereof), fibroblast growth factor-8 (FGF8) (or functional fragments thereof), or combinations thereof. Additionally, the cells are preferably plated on a surface that permits adhesion of CNS stem cells, such as a fibronectin-, laminin- or vitronectin-coated surface.

In the fifth stage, the expanded CNS precursor cells are differentiated to form neuronal cells. The differentiation is induced by withdrawal of at least one neurologic agent, preferably by the withdrawal of bFGF and/or EGF. Generally, the differentiation media is similar to the proliferation media used in stage four (but without bFGF or EGF). Additionally, the differentiation media may also include factors to enhance differentiation into midbrain neurons, for example, ascorbic acid (AA).

In one embodiment, the ES cells are transfected with a gene encoding Nurr1. The Nurr1-transfected cells are then differentiated using the method of the invention. Generally, Nurr1 transfected cells differentiated according to the method of the invention generate 2 to 10 fold, more typically 4 to 5 fold more dopaminergic cells, when compared to the number of dopaminergic cells generated by differentiating wild-type ES cells using the method of the invention. Additionally, Nurr1 transfected cells differentiated according to the method of the invention generate cells that produce 50 to 5000 times, more typically 100 to 1000 times more dopamine than wild-type ES cells differentiated according to the method of the invention. In yet another embodiment, the ES cells can be transfected with other genes of the steroid/thyroid hormone nuclear receptor superfamily, more preferably genes of the NGFI-B subfamily, for examples genes such as Pxt3, Nurr77, or NGFI-B.

The method of the invention can be used to culture a variety of cells, preferably neuronal cells, including, but not limited to dopaminergic neuron cells, cholinergic neuronal cells and serotonergic neuron cells. Preferably, the method of the invention is used to generate dopaminergic and serotonergic cells. Most preferably, the method is used to generated dopaminergic cells.

The invention also provides a method for treating a neurological disorder, such as Parkinson's disease, a method of introducing a gene product into a brain of a patient, and an assay for neurologically active substances.

The invention also provides a cell culture which includes about 50% to about 85%, more typically between about 65% and 80% neurons. Of the neurons in the cell culture, typically between about 20% and 40%, more typically between about 25% and 30% are dopaminergic neurons. The cell culture also includes glial cells, typically between about 1% to about 3% astrocytes. Typically, at least some (e.g., at least 90%, more preferably at least 95%) of the neuronal cells in the culture are synaptically active.

### BRIEF DESCRIPTION OF THE FIGURES

FIG. 1 is a flow chart for a process of generating dopamine neuronal populations from undifferentiated ES cells

FIG. 2 is a photograph of a gel showing differential gene expression in the cells in stages 1-4 of the process outlined in FIG. 1.

FIG. 3 is a photograph of a gel showing that components of SHH and FGF8 signaling pathways are expressed during ES cell development.

FIG. 4 is a bar graph showing the combination of SHH and FGF8 during stage 4 and ascorbic acid during stage 5 increase the yield of TH+ neurons in ES cell cultures.

FIG. 5 is a bar graph demonstrating that the ES cells-derived cells secrete dopamine.

FIG. 6 is a bar graph showing that SHH promotes generation of serotonergic neurons.

FIG. 7 is a bar graph comparing the amount of TH+ and TUJ1+ cells produced using the method of the invention and the method of Okabe et al.

FIG. 8 is a bar graph comparing the amount of TH+ cells produced using various culture media.

FIG. 9A is a graph showing the action potential spiking behavior of differentiated neurons within a cell culture of the invention.

FIG. 9B is a graph showing the action potential after application of GABA to the dendrites of an ES-derived neuron.

FIG. 9C is a graph showing the action potential after application of glutamate to the dendrites of an ES-derived neuron.

FIG. 9D is a graph showing a decrease in spontaneous activity in an ES-derived neurons when action potentials were blocked with tetrodotoxin.

FIG. 10A is a graph comparing TuJ1 expression of differentiated wild-type ES cells and Nurr1-transfected cells.

FIG. 10B is a graph comparing dopamine relase from differentiated wild-type cells and Nurr1-transfected ES cells.

FIG. 11A shows two graphs comparing the rotational behavior of animals grafted with differentiated wild-type cells and Nurr1-transfected ES cells.

FIG. 11B is a patch clamp recordings showing the synaptic activity of differentiated Nurr1-transfected ES cells.

### DETAILED DESCRIPTION OF THE INVENTION

A. Overview

A first aspect of the invention provides a technique for generating specific neuronal cell types, in particular, neurons with patterns of gene expression characteristic of midbrain neurons. In a preferred embodiment, the invention provides a technique for culturing embryonic stem (ES) cells to provide dopaminergic neurons. However, the process may also be used to generate other neuronal subtypes, for example, ES derived GABAergic, glutamertegic, cholinergic, and serotonergic neurons. Preferably, the method is used to generate ventral neurons such as cholinergic, dopaminergic and serotonergic neurons. Dopaminergic and serotonergic neurons are derived from a common progenitor cells. The cell culture technique preferably generates ventral neuron subtypes derived from this common progenitor, such as dopaminergic and serotonergic neurons. Furthermore, cells of other mid and/or hindbrain structures, such as the cerebellum, may be derived from ES cells using the process of the invention.

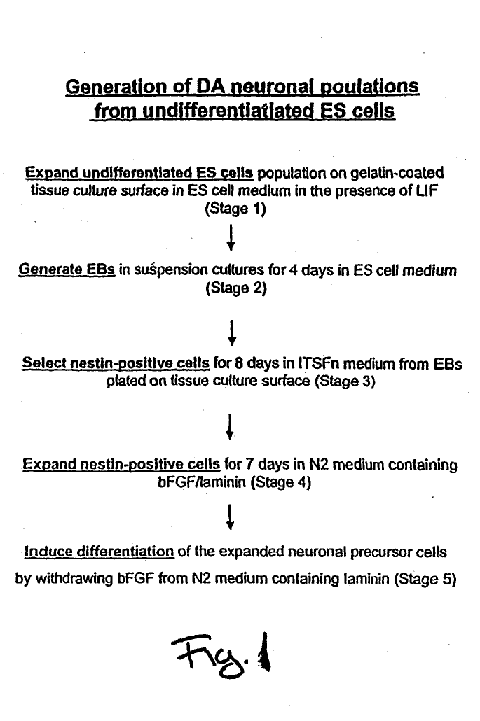

According to the invention, specific culture conditions induce the progression of ES cells through a series of transitions that culminate in the generation of functional differentiated neurons. The strategy for inducing nervous system differentiation of ES cells is shown schematically in FIG. 1. In the first stage (stage 1), undifferentiated ES cells are expanded. In the second stage (stage 2), embryoid bodies that include an inner core of undifferentiated stem cells surrounded by primitive endoderm are generated in suspension culture. Preferably, the embryoid bodies are generated from individual ES cells, rather than clusters of ES cells. In the third step (stage 3), the cells of the embryoid bodies are cultured to select for Central Nervous System (CNS) precursor (or stem) cells. In the subsequent step (stage 4), the CNS precursors are expanded in the presence of neurologic agents which encourage the formation of midbrain neuronal precursors, preferably dopaminergic neuronal precursors. Examples of neurologic agents include basic fibroblast growth factor (bFGF), fibroblast growth factor-8 (FGF8) and sonic hedgehog (SHH) protein. The inventors have discovered that culturing CNS precursors in media which includes FGF8 and SHH enhances the generation of midbrain (e.g., dopaminergic) neurons. Differentiation of the expanded CNS precursors is then induced by the withdrawal of at least one neurologic agent, typically bFGF (stage 5). Preferably, the CNS precursors are also exposed to ascorbic acid during differentiation (stage 5). According to the invention, the CNS precursors differentiate into midbrain neuron cells. Preferably, the expanded CNS precursors are cultured in a differentiation media that includes ascorbic acid. In a preferred embodiment, approximately 20% to 40% of the neuronal cells are dopamine neurons.

According to the process of the invention, a culture of between about 1×10⁶ to about 5×10⁶ ES cells typically generate between about 7×10⁶ to about 35×10⁶ neurons. Preferably, the culture includes between about 2×10⁶ to about 15×10⁶ dopaminergic neurons, i.e., about 2 to about 3 dopaminergic neurons are harvested (stage 5) for every undifferentiated ES cell plated (stage 1).

Another aspect of the invention is directed to a neuronal cell culture which includes about 50% to about 85%, more typically between about 65% and 80% neurons. Of the neurons in the cell culture, typically between about 20% and 40%, more typically between about 25% and 30% are dopaminergic neurons (TH+ cells). The cell culture also includes about 1% to about 3% astrocytes.

The percentage of dopaminergic neurons obtained in the process of the invention is higher than any other process known at this time, in vitro or in vivo. Generally, the efficiency of dopamine neuron generation has not surpassed 5% of total cells in primary mesencephalic cultures (Spenger et al., (1996) “Fetal ventral mesencephalon of human and rat origin maintained in vitro and transplanted to 6-hyrosydopamine-lesioned rats gives rise to grafts rich in dopaminergic neurons,” Exp. Brain. Res. 112:47-57) and 18.3% of total cells in cultures generated from bFGF expanded midbrain precursors. (Studer et al., (1998) “Transplantation of expanded mesencephalic precursors leads to recovery in parkinsonian rats,” Nature Neurosci. 1:290-295).

B. Definitions

“Fibroblast growth factor” or “FGF” refers to any suitable fibroblast growth factor, derived from any animal, and functional fragments thereof. A variety of FGF's are known and include, but are not limited to, FGF-1 (acidic fibroblast growth factor), FGF-2 (basic fibroblast growth factor), FGF-3 (int-2), FGF-4 (hst/K-FGF), FGF-5, FGF-6, FGF-7, FGF-8, FGF-9 and FGF-98.

“Central Nervous System” or “CNS” refers to the part of the nervous system of an animal that contains a high concentration of cell bodies and synapses and is the main site of integration of nervous activity. In higher animals, the CNS generally refers to the brain and spinal cord.

As used herein, the term “differentiation” refers to the process whereby relatively unspecialized cells (e.g., embryonic cells) acquire specialized structural and/or functional features characteristic of mature cells. Typically, during differentiation, cellular structure alters and tissue-specific proteins appear. The term “differentiated neuronal cells” refers to cells expressing a full complement of proteins characteristic of the specific neuronal cell type in contrast to other nerve cell types, such as astrocytes, oligodendrocytes and glial cells.

“Dopaminergic neurons” refers to neuronal cells that produce the neurotransmitter dopamine. Typically, dopaminergic neurons are highly concentrated in the substantia nigra of the midbrain.

Dopamine, along with epinephrine, norepinephrine, and serotonin, belongs to a chemical family referred to “monoamines.” Within the family of monoamines, epinephrine, norepinephrine, and dopamine are derived from the amino acid tyrosine and form a subfamily called the catecholamines. Frequently, tyrosine hydroxylase (TH), the rate-limiting enzyme for the biosynthesis of dopamine, is used as a marker to identify dopaminergic neurons.

An “effective amount” of agent is an amount sufficient to prevent, treat, reduce and/or ameliorate the symptoms and/or underlying causes of any of the above disorders or diseases. In some instances, an “effective amount” is sufficient to eliminate the symptoms of those diseases and, perhaps, overcome the disease itself.

“Embryonic stem (ES) cells” refers to cells isolated from the inner cell mass of the developing blastocyst. “ES cells” can be derived from any organism. ES cells derived from mammals, including mice, rats, rabbits, guinea pigs, goats, pigs, cows and humans. Human and murine derived ES cells are preferred. ES cells are totipotent cells, meaning that they can generate all of the cells present in the body (bone, muscle, brain cells, etc.).

As used herein, the terms “expand”, “expansion” or expanded” refer to a process by which the number or amount of cells in a cell culture is increased due to cell division. The terms “proliferate”, “proliferation” or “proliferated” may be used interchangeably with the words “expand”, “expansion”, or “expanded.” Typically, during an expansion phase, the cells do not differentiate to form mature cells.

The term “neurological disorder” refers to a disorder in the nervous system, including the CNS and PNS. Examples of neurological disorders include Parkinson's disease, Huntington's disease, Alzheimer's disease, severe seizure disorders including epilepsy, familial dysautonomia as well as injury or trauma to the nervous system, such as neurotoxic injury or disorders of mood and behavior such as addiction, schizophrenia and amyotrophic lateral sclerosis.

The term “patient” as used herein generally refers to any warm blooded mammal, such as humans, non-human primates, rodents and the like which is to be the recipient of the particular treatment.

“Peripheral Nervous System” or “PNS” refers to the part of an animal's nervous system other than the Central Nervous System. Generally, the PNS is located in the peripheral parts of the body and includes cranial nerves, spinal nerves and their branches, and the autonomic nervous system.

“Precursor” or “stem” cell refers to a cell that can generate a fully differentiated functional cell of a given cell type. The role of stem cells in vivo is to replace cells that are destroyed during the normal life of an animal. Generally, stem cells can divide without limit. After division, the stem cell may remain as a stem cell or proceed to terminal differentiation. Although appearing morphologically unspecialized, the stem cell may be considered differentiated where the possibilities for further differentiation are limited.

“Prevent”, as used herein, refers to putting off, delaying, slowing, inhibiting, or otherwise stopping, reducing or ameliorating the onset of such brain diseases or disorders.

Sonic Hedgehog (SHH), Desert Hedgehog (DHH), and Indian Hedgehog (IHH) genes encode a family of morphogen proteins that are implicated in a wide range of signaling activities, particularly during embryonic development. These secreted proteins are proposed to mediate their effects on target cells by interacting with their putative receptor, Patched (Ptc), and with a seven-pass transmembrane protein, Smoothened (Smo). However, the roles that these signaling molecules may play in adult tissues, particularly in brain, are not yet clearly defined. Data suggests that, besides its roles in determining cell fate and patterning during embryogenesis, the hedgehog signaling pathway may have also important roles in the adult brain. “Sonic Hedgehog Protein” or “SHH” refers to any suitable sonic hedgehog protein, derived from any animal, and functional fragments thereof.

“Synapse” refers to highly specialized intercellular junctions between neurons and between neurons and effector cells across which a nerve impulse is conducted. Generally, the nerve impulse is conducted by the release from one neuron (pre-synaptic neuron) of a chemical transmitter (such as dopamine or serotonin), which diffuses across the narrow intercellular space to the other neuron or effector cell (post-synaptic neuron). Generally neurotransmitters mediate their effects by interacting with specific receptors incorporated in the post-synaptic cell. “Synaptically active” refers to cells (e.g., differentiated neurons) which receive and transmit action potentials characteristic of mature neurons.

The term “full-length” peptide refers to the peptide encoded by the full DNA coding sequence. The full-length peptide can be either a wild-type or a mutant peptide.

The term “wild-type” refers to a naturally occurring phenotype that is characteristic of most of the members of a species (in contrast to the phenotype of a mutant, such as a mutant created by genetic modification).

The term “mutant” refers to a peptide not having a wild-type sequence. The term “mutein” refers to a mutant protein produced by site-specific mutagenesis or other recombinant DNA technique wherein the mutein retains the desired activity of the wild-type peptide. Preferred mutants include mutants containing conservative amino acid substitutions. As used herein, “conservative amino acid substitution” refers to a replacement of one or more amino acid residue with a different residue having a sidechain with at least one similar biochemical characteristic, such as size, shape, charge or polarity.

The term “fragment” refers to a sequence that includes at least part of the wild-type sequence or mutant sequence, wherein the fragment retains the desired activity of the wild-type peptide. Preferably, the DNA or RNA encoding the fragment or mutant is capable of hybridizing to all or a portion of the DNA or RNA of the wild-type protein, or its complement, under stringent or moderately stringent hybridization conditions (as defined herein).

The term “hybridizing” refers to the pairing of complementary nucleic acids. Hybridization” can include hydrogen bonding, which may be Watson-Crick, Hoogsteen or reversed Hoogsteen hydrogen bonding, between complementary nucleoside or nucleotide bases. Hybridization and the strength of hybridization (i.e., the strength of the association between the nucleic acids) is influenced by such factors as the degree of complementarity between the nucleic acids, stringency of the conditions involved, the melting temperature (Tₘ) of the formed hybrid, and the G:C ratio within the nucleic acids. Complementarity may be “partial,” in which only some of the bases of the nucleic acids are matched according to the base pairing rules. Alternatively, there may be “complete” or “total” complementarity between the nucleic acids. The degree of complementarity between the nucleic acid strands has effects on the efficiency and strength of hybridization between the nucleic acid strands.

As used herein, the term “percent homology” or “percent identity” of two nucleic acid sequences is determined using the algorithm of Karlin and Altschul (1990) Proc. Natl. Acad. Sci. USA 87: 2264-2268, modified as in Karlin and Altschul (1993) Proc. Natl. Acad. Sci. USA 90: 5873-5877. Such an algorithm is incorporated into the NBLAST program of Altschul et al. (1990) J. Mol. Biol. 215: 402-410. To obtain gapped alignments for comparision purposes, Gapped BLAST is used as described by Altschul et al. (1997) Nuelic Acids Res. 25: 3389-3402. When using BLAST and gapped BLAST programs, the default parameters of the respective programs (e.g., NBLAST) are used.

“Percent (%) amino acid sequence identity” is defined as the percentage of amino acid residues in a candidate sequence that are identical with the amino acid residues in the wild-type sequence after aligning the sequences in the same reading frame and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST software. Those skilled in the art can determine appropriate parameters for measuring alignment, including any algorithms needed to achieve maximal alignment over the full length of the sequence being compared.

“Percent (%) nucleic acid sequence identity” with respect to the wild-type sequences identified herein is defined as the percentage of nucleotides in a candidate sequence that are identical with the nucleotides in the sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software. Those skilled in the art can determine appropriate parameters for measuring alignment, including any algorithms needed to achieve maximal alignment over the full length of the sequences being compared.

“Treat”, “Treating”, “Treatment” and “Therapy” refer to any one or more of reducing or eliminating the symptoms of a particular disorder, slowing the progression, attenuating or curing an existing disease.

C. Neurologic Agents

As used herein, the term “neurologic agent” refers to any substance that promotes the function or survival of neurons. For example, a preferred neurologic agent can promote nerve or glial cell growth, promote survival of functioning cells, augment the activity of functioning cells, enhance the synthesis of neurotransmitter substances, augment the activity of naturally occurring nerve growth promoting factors, act as a nerve growth promoting factor, prevent degeneration of neurons, induce regrowth of dendrite and axon, have more than one of these properties, or the like. A preferred neurologic agent is a neurotrophic and/or neuritogenic factor that is similar to a naturally occurring nerve growth promoting substance. Numerous neurologic agents are known to those of skill in the art.

The term “neurologic agent” includes nerve growth factor (NGF), neurotrophins such as neurotrophins 3, 4, and/or 5 (NT-3, NT-4 and/or NT-5), brain-derived neurotrophic factor (BDNF), fibroblast growth factors (FGFs, e.g., basic fibroblast growth factor), insulin, insulin-like growth factors (IGFs, e.g., IGF-I and/or IGF-II), ciliary neurotrophic factor (CNTF), glia-derived neurotrophic factor (GDNF), glia-derived nexin, hedgehog proteins, including sonic hedgehog (SHH), desert hedgehog (DHH) and Indian hedgehog (IHH), Nurr1, Ptx3, Nurr77 and NGFI-B, combinations thereof, and functional fragments thereof.

1. FGF

“FGF” refers to a fibroblast growth factor protein such as FGF-1, FGF-2, FGF-4, FGF-6, FGF-8, FGF-9 or FGF-98, or a biologically active fragment or mutein thereof. The FGF can be from any animal species including, but not limited to, rodent, avian, canine, bovine, porcine, equine, and, preferably, human. Preferably the FGF is from a mammalian species, and more preferably is from a mammal of the same species as the mammal undergoing treatment.

The amino acid sequences and method for making many of the FGFs are well known in the art.

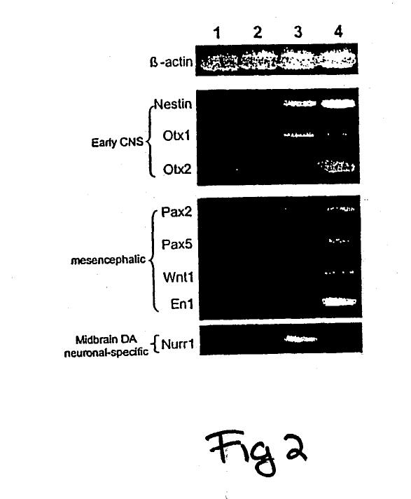

The amino acid sequence of human FGF-1 and a method for its recombinant expression are disclosed in U.S. Pat. No. 5,604,293 (Fiddes), entitled “Recombinant Human Basic Fibroblast Growth Factor,” which issued on Feb. 18, 1997. See FIG. 2d of the '293 patent. The amino acid sequence of bovine FGF-1 is disclosed in U.S. Pat. No. 5,604,293 (Fiddes) at FIG. 1b, as is a method for its expression. The mature forms of both human FGF-1 and bovine FGF-1 have 140 amino acid residues, differing only at 19 residue positions.

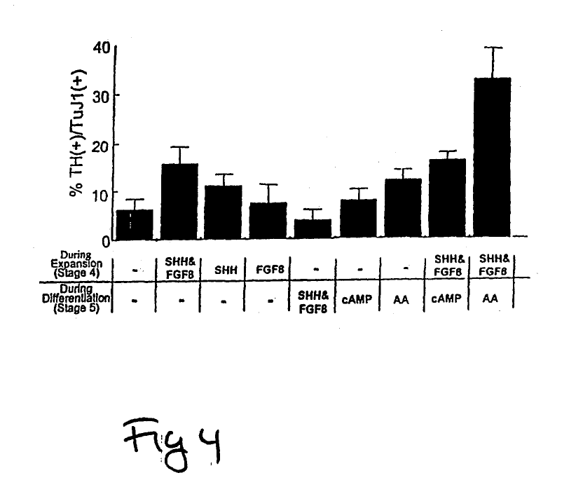

The amino acid sequence of human FGF-2 and methods for its recombinant expression are disclosed in U.S. Pat. No. 5,439,818 (Fiddes) entitled “DNA Encoding Human Recombinant Basic Fibroblast Growth Factor,” which issued on Aug. 8, 1995 (see FIG. 4 therein). The amino acid sequence of bovine FGF-2 and various methods for its recombinant expression are disclosed in U.S. Pat. No. 5,155,214, entitled “Basic Fibroblast Growth Factor,” which issued on Oct. 13, 1992. When the 146 residue forms are compared, their amino acid sequences are nearly identical with only two residues that differ.

FGF-3 was first identified as an expression product of a mouse int-2 mammary tumor and its amino acid sequence is disclosed in Dickson et al., “Potential Oncogene Product Related to Growth Factors,” Nature 326:833 (Apr. 30, 1987). FGF-3 which has 243 residues when the N-terminal Met is excluded, is substantially longer than both FGF-2 and FGF-2.

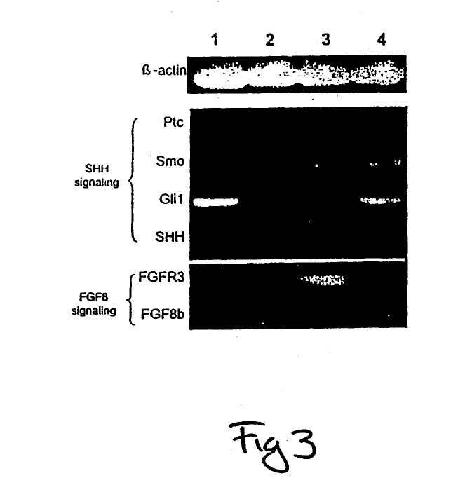

The amino acid sequence for human FGF-4 (previously referred to as “hst”), was first disclosed by Yoshida, et al., “Genomic Sequence of hst, a Transforming Gene Enclosing a Protein Homologous to Fibroblast Growth Factors and the int-2 Enclosed Protein,” PHAS USA, 84:7305-7309 (October 1987) at FIG. 3. Including its leader sequence, FGF-4 has 206 amino acid residues. When the amino acid sequences of human FGF-4, FGF-1, FGF-2 and murine FGF-3 are compared, residues 72-204 of human FGF-4 have 43% homology to human FGF-2; residues 79-204 have 38% homology to human FGF-1; and residues 72-174 have 40% homology to murine FGF-3. A comparison of these four sequences in overlap form is shown in Yoshida (1987) at FIG. 3.

The cDNA and deduced amino acid sequences for human FGF-5 are disclosed in Zhan, et al., “The Human FGF-5 Oncogene Encodes a Novel Protein Related to Fibroblast Growth Factors,” Molec. And Cell. Biol., 8(8):3487-3495 (August 1988) at FIG. 1. A comparison between the amino acid sequences of human FGF-1, human FGF-2, murine FGF-3, human FGF-4 and FGF-5 is presented in FIG. 2 of Zhan (1988). In FIG. 2 of Zhan, human FGF-1, FGF-2, and FGF-4 and murine FGF-3 are identified as aFGF (i.e., acidic FGF), bFGF (i.e., basic FGF), and hstKS3 and int-2, respectively. In the above referenced comparison, two blocks of FGF-5 amino acid residues (90 to 180 and 187-207) showed substantial homology to FGF 1-4, i.e., 50.4% with FGF-4, 47.5% with FGF-3, 43.4% with FGF-2 and 40.2% with FGF-1.

The cDNA and deduced amino acid sequence for human FGF-6 are disclosed in Coulier et al., “Putative Structure of the FGF-6 Gene Product and Role of the Signal Peptide,” Oncogene 6:1437-1444 (1991) at FIG. 2. Coulier also discloses a method for cloning FGF-6. FGF-6 is one of the largest of the FGFs, having 208 amino acid residues. When the amino acid sequences of human FGF-1, FGF-2, FGF-3, FGF-4, FGF-5, FGF-6 and FGF-7 are compared, there are strong similarities in the C-terminal two-thirds of the molecules (corresponding e.g., to residues 78-208 of human FGF-6. In particular, 23 residues of FGF-6, including the two cysteines at residue positions 90-157 of FGF-6 were identical between the seven members of the family. This number increases to 33 residues when conserved amino acid residues are considered. The overall similarities between these seven human FGFs ranged from 32% to 70% identical residues and 48% to 79% conserved residues for the C-terminal two-thirds of the molecules.

The amino acid sequence of human FGF-7 is well-known in the art and disclosed in Miyamoto, et.al., “Molecular Cloning of a Novel Cytokine cDNA Encoding the Ninth Member of the Fibroblast Growth Factor Family, Which has a Unique Secretion Property,” Mol. And Cell. Biol. 13(7):4251-4259 (1993) at FIG. 2. In Miyamoto, human FGF-7 was referred to by its older name “KGF”. FGF-7 has 191 amino acids.

The cDNA and deduced amino acid sequence of murine FGRF-8 is well-known in the art and disclosed in Tanaka et. A., “Cloning and Characterization of an Androgen-Induced Growth Factor Essential for the Growth of Mouse Mammary Caricnoma Cells,” PNAS USA, 89:8928-8932 (1992) at FIG. 2. Tanaka also discloses a method for making recombinant FGF-8. The FGF-8 of Tanaka has 215 amino acid residues. MacArthur, et al., “FGF-8 isoforms activate receptor splice forms that are expressed in mesenchymal regions of mouse development,” Development, 1212:3603-3613 (1995) discloses the FGF-8 has 8 different insoforms that differ at the mature N-terminus but that are identical over the C-terminal region. The 8 isoforms arise because FGF-8 has 6 exons of which the first four (which correspond to the first exon of most other FGF genes) result in alternative splicing.

The cDNA and deduced amino acid sequences of human and murine FGF-9 are known in the art and methods for their recombinant expressions are disclosed in Santos-Ocamp, et. Al., “Expression and Biological Activity of Mouse Fibroblast Growth Factor,” J. Biol. Chem., 271(3):1726-1731 (1996). Both the human and murine FGF-9 molecules have 208 amino acid residues and sequences that differ by only two residues.

The cDNA and amino acid sequence of human FGF-98 and a method for its recombinant expression are disclosed in provisional patent application Serial No. 60/083,553 which is hereby incorporated herein by reference in its entirety. FGF-98, which is also known as FGF-18, has 207 amino acid residues.

bFGF-2, and other FGFs, can be made as described in U.S. Pat. No. 5,155,214 (“the '214 patent”). The recombinant bFGF-2, and other FGFs, can be purified to pharmaceutical quality (98% or greater purity) using the techniques described in detail in U.S. Pat. No. 4,956,455 (the '455 patent), entitled “Bovine Fibroblast Growth Factor” which issued on Sep. 11, 1990.

Biologically active variants of FGF are also encompassed by the method of the present invention. Such variants should retain FGF activities, particularly the ability to bind to FGF receptor sites. FGF activity may be measured using standard FGF bioassays, which are known to those of skill in the art. Representative assays include known radioreceptor assays using membranes, a bioassay that measures the ability of the molecule to enhance incorporation of tritiated thymidine, in a dose-dependent manner, into the DNA of cells, and the like. Preferably, the variant has at least the same activity as the native molecule.

In addition to the above described FGFs, the neurologic agent also includes an active fragment of any one of the above-described FGFs. In its simplest form, the active fragment is made by the removal of the N-terminal methionine, using well-known techniques for N-terminal Met removal, such as a treatment with a methionine aminopeptidase. A second desirable truncation includes an FGF without its leader sequence. Those skilled in the art recognize the leader sequence as the series of hydrophobic residues at the N-terminus of a protein that facilitate its passage through a cell membrane but that are not necessary for activity and that are not found on the mature protein.

Preferred truncations on the FGFs are determined relative to mature FGF-2 having 146 residues. As a general rule, the amino acid sequence of an FGF is aligned with FGF-2 to obtain maximum homology. Portions of the FGF that extend beyond the corresponding N-terminus of the aligned FGF-2 are generally suitable for deletion without adverse effect. Likewise, portions of the FGF that extend beyond the C-terminus of the aligned FGF-2 are also capable of being deleted without adverse effect.

Fragments of FGF that are smaller than those described can also be employed in the present invention.

Suitable biologically active variants can be FGF analogues or derivatives. By “analogue” is intended an analogue of either FGF or an FGF fragment that includes a native FGF sequence and structure having one or more amino acid substitutions, insertions, or deletions. Analogs having one or more peptoid sequences (peptide mimic sequences) are also included (see e.g. International Publication No. WO 91/04282). By “derivative” is intended any suitable modification of FGF, FGF fragments, or their respective analogues, such as glycosylation, phosphorylation, or other addition of foreign moieties, so long as the FGF activity is retained. Methods for making FGF fragments, analogues, and derivatives are available in the art.

In addition to the above described FGFs, the method of the present invention can also employ an active mutein or variant thereof. By the term active mutein, as used in conjunction with an FGF, is meant a mutated form of the naturally occurring FGF. FGF muteins or variants will generally have at least 70%, preferably 80%, more preferably 85%, even more preferably 90% to 95% or more, and most preferably 98% or more amino acid sequence identity to the amino acid sequence of the reference FGF molecule. A mutein or variant may, for example, differ by as few as 1 to 10 amino acid residues, such as 6-10, as few as 5, as few as 4, 3, 2, or even 1 amino acid residue.

The sequence identity can be determined as described hereinabove. For FGF, a preferred method for determining sequence identify employs the Smith-Waterman homology search algorithm (Meth. Mol. Biol. 70:173-187 (1997)) as implemented in MSPRCH program (Oxford Molecular) using an affine gap search with the following search parameters: gap open penalty of 12, and gap extension penalty of 1. Preferably, the mutations are “conservative amino acid substitutions” using L-amino acids, wherein one amino acid is replaced by another biologically similar amino acid. As previously noted, conservative amino acid substitutions are those that preserve the general charge, hydrophobicity, hydrophilicity, and/or steric bulk of the amino acid being substituted. Examples of conservative substitutions are those between the following groups: Gly/Ala, Val/Ile/Leu, Lys/Arg, Asn/Gln, Glu/Asp, Ser/Cys/Thr, and Phe/Trp/Tyr.

One skilled in the art, using art known techniques, is able to make one or more point mutations in the DNA encoding any of the FGFs to obtain expression of an FGF polypeptide mutein (or fragment mutein) having angiogenic activity for use in method of the present invention. To prepare an biologically active mutein of an FGF, one uses standard techniques for site directed mutagenesis, as known in the art and/or as taught in Gilman, et al., Gene, 8:81 (1979) or Roberts, et al., Nature, 328:731 (1987), to introduce one or more point mutations into the cDNA that encodes the FGF.

2. Nurr1

Nurr1 (also known as NOT/TINUR/RNR-1/HZF-3) is an orphan nuclear receptor, a member of the steroid/thyroid hormone nuclear receptor superfamily, in the NGFI-B subfamily, which also includes NOR1. Nurr1 is predominantly expressed in the midbrain; substantia nigra (SN) and ventral tegmental area (VTA). Nurr1 is associated with the development and differentiation of midbrain DA neurons.

Human Nurr1 gene has been mapped on chromosome 2q22-23. The sequence for human Nurr1, which is approximately 8.3 kb long, consisting of eight exons and seven introns has been characterized by Torii et al. “Organization of the human orphan receptor Nurr1 gene,” Gene 230(2):225-32 (1999); and Ichinose et al., “Molecular cloning of the human Nurr1 gene: characterization of the human gene and cDNAs,” Gene 230(2):233-9 (1999). The murine Nurr1 gene is approximately 7 kb long and has been characterized by Law et al., “Identification of a new brain-specific transcription factor, NURR1,” Mol. Endocrinol. 6(12):2129-2135 (1992); and Castillo et al., “Organization, sequence, chromosomal localization, and promoter identification of the mouse orphan nuclear receptor Nurr1 gene,” Genomics 41(2):250-257 (1997).

Human Nurr1, mouse Nurr1, mouse Nur77 and human NOR-1 have been shown to have highly conserved genomic structures.

As used herein, the term “Nurr1” not only refers to the full-length, wild-type Nurr1 sequence, but also mutants, and fragments of the wild-type and mutant sequences. That is, the method of the invention can be used with a fragment of Nurr1 protein or a mutant thereof. Variants of Nurr1 preferably retain Nurr1 activity.

An active fragment or variant of Nurr1 can be used. Preferred fragments and variants retain the DNA binding domain of the protein.

Suitable biologically active variants can be Nurr1 analogues or derivatives. By “analogue” is intended an analogue of either Nurr1 or a Nurr1 fragment that includes a native Nurr1 sequence and structure having one or more amino acid substitutions, insertions, or deletions. Analogs having one or more peptoid sequences (peptide mimic sequences) are also included (see e.g. International Publication No. WO 91/04282). By “derivative” is intended any suitable modification of Nurr1, Nurr1 fragments, or their respective analogues, such as glycosylation, phosphorylation, or other addition of foreign moieties, so long as the Nurr1 activity is retained. Methods for making Nurr1 fragments, analogues, and derivatives are available in the art.

Additionally, an active mutein or variant of Nurr1 can be used. The term mutein refers to a mutated form of a naturally occurring Nurr1. Preferably Nurr1 muteins or variants have at least 70%, preferably 80%, more preferably 85%, even more preferably 90% to 95% or more, and most preferably 98% or more amino acid sequence identity to the amino acid sequence of the reference Nurr1 molecule. A mutein or variant may, for example, differ by as few as 1 to 10 amino acid residues, such as 6-10, as few as 5, as few as 4, 3, 2, or even 1 amino acid residue. The sequence identity can be determined as described hereinabove. Preferably, the mutations are “conservative amino acid substitutions” using L-amino acids, wherein one amino acid is replaced by another biologically similar amino acid. As previously noted, conservative amino acid substitutions are those that preserve the general charge, hydrophobicity, hydrophilicity, and/or steric bulk of the amino acid being substituted. Examples of conservative substitutions are those between the following groups: Gly/Ala, Val/Ile/Leu, Lys/Arg, Asn/Gln, Glu/Asp, Ser/Cys/Thr, and Phe/Trp/Tyr.

One skilled in the art, using art known techniques, is able to make one or more point mutations in the DNA encoding Nurr1 to obtain expression of Nurr1 mutein (or fragment mutein) having activity for use in method of the present invention.

Preferably hybridizing portion of the hybridizing nucleic acids is at least 15 (e.g., 20, 25, 30 or 50) nucleotides in length and at least 50% (e.g., at least 80%, 95%, or 98%) identical to a sequence of wild type Nurr1, or its complement, or fragments thereof.

Although the disclosure focuses on transfection with Nurr1, other members of the steroid/thyroid hormone nuclear receptor family, particularly in the NGFI-B subfamily can be used, for example Ptx3, Nurr77 and NGFI-B.

D. Process for Obtaining Differentiated Midbrain Neural Cells from Embryonic Stem Cells

The invention provides a technique for generating of functional dopamine neurons from embryonic stem cells. The strategy for inducing nervous system differentiation of ES cells (shown schematically in FIG. 1) is similar to that introduced by Okabe et al., (1996) “Development of neuronal precursor cells and functional postmitotic neurons from embryonic stem cells in vitro,” Mech. Dev. 59:89-102.

1. Expansion of Undifferentiated Embryonic Stem (ES) Cells

In the first stage (stage 1), undifferentiated embryonic stem (ES) cells, for example human, mouse or rat ES cells, are cultured in ES proliferation media to expand the number of cells. Generally, the ES cells can be expanded at least about 1000 fold without losing pluripotency.

Preferably, ES cells are cultured in an ES growth media which generally includes a carbon source, a nitrogen source and a buffer to maintain pH. More specifically, ES growth media typically contains a minimal essential media, such as DMEM, supplemented with various nutrients to enhance ES cell growth. Additionally, the minimal essential media may be supplemented with additives such as horse, calf or fetal bovine serum (between about 10% by dry weight and 15% by dry weight), nonessential amino acids, L-glutamine, and antibiotics such as streptomycin, penicillin, and combinations thereof. 2-mercaptoethanol may also be included in the media. ES growth media is commercially available, for example as KO-DMEM (Life-Tech Catalog No. 10829-018).

Other methods and media for obtaining and culturing embryonic stem cells are known and are suitable for use in this invention, see, for example, Evans et al., (1981), “Establishment in culture of pluripotential cells from mouse embryos,” Nature 292:154-156; Martin et al., (1981) “Isolation of a pluripotent cell line from early mouse embryos cultured in medium conditioned by teracarcinoma stem cells,” Proc. Natl. Acad. Sci. 78:7634-7636; Robertson et al., (1986) “Germ line transmission of a gene introduced into cultured pluripotential cells by, a retroviral vector,” Nature 323:445-448; Doetschman et al., (1987) “Targeted correction of a mutant HPRT gene in mouse embryonic stem cells,” Nature 330:576-578; “Thomas et al., (1987) “Site directed mutagenesis by gene targeting in mouse embryo-derived stem cells,” Cell 51:503-512; Thomson et al., (1998) “Embryonic stem cell lines derived from human blastocysts,” Science 282:1145-1147; and Shamblott et al., (1998) “Derivation of pluripotent stem cells from cultured human primordal germ cells,” Proc. Natl. Acad. Sci. U.S.A. 95:13726-13731. The disclosures of these seven references are hereby incorporated by reference herein.

Preferably, the ES cells are cultured on plates, for example surface coated plates, which prevent differentiation of the ES cells, such as gelatin coated tissue culture plates or on plate which includes a feeder cell layer such as a fibroblast cell layer in the presence of LIF (Leukemia Inhibitory Factor), a growth factor that prevents differentiation of ES cells. Generally, the ES cells are cultured for about 4 days to about 8 days, more preferably about 6 days to about 7 days at a temperature between about 35° C. and about 40° C., more preferably about 37° C. under an atmosphere which contains oxygen and between about 1 wt % to 5 wt % CO₂. Preferably, the media is changed about every 1 to 2 days.

Although an expansion step is not necessary, the ES cells are preferably expanded as described above prior to the stage of embryoid body formation (stage 2) to increase the amount of differentiated midbrain neurons produced by the process of the invention.

2. Generation of Embryoid Bodies

In the second stage (stage 2), embryoid bodies are generated in suspension culture according to the method described by Martin et al., (1975) “Differentiation of clonal lines of teratocarcinoma cells: Formation of embryoid bodies in vitro,” Proc. Natl. Acad. Sci. 72:1441-1445).

Briefly, to form embryoid bodies, the clusters of ES cells are disengaged from the tissue culture plates. Methods for disengaging cells from tissue culture plates are known and include the use of enzymes, such as trypsin or papain, or commercially available preparations.

Generally, the ES cells disengage from the tissue culture plates in clusters (e.g., aggregates of 10 or more ES cells, typically 50 or more cells). The clusters of ES cells are then dissociated to obtain a population of cells which includes a majority of (e.g., between 50% and 70%, more preferably between 75% and 100%) individual cells. Methods for dissociating clusters of cells are likewise known. One method for dissociating cells includes mechanically separating the cells, for example, by repeatedly aspirating a cell culture with pipet. Preferably, the ES cells are in an exponential growth phase at the time of dissociation to avoid spontaneous differentiation that tends to occurs in an overgrown culture.

The dissociated ES cells are then cultured in ES proliferation media, described above. However, in contrast to the ES cell proliferation stage (in which the cells are grown on a tissue culture dish surface), the embryoid bodies are generated in suspension. For example, the cells may be cultured on non-adherent bacterial culture dishes. In this stage, the cells are incubated for about 4 days to about 7 days. Preferably, the medium is changed every 1 to 2 days.

As used herein, the term “embryoid bodies” refers to ES cell aggregates generated when ES cells are plated on a non-adhesive surface that prevents attachment and differentiation of the ES cells. Generally, embryoid bodies include an inner core of undifferentiated stem cells surrounded by primitive endoderm.

3. Selection for CNS Precursors

In the third step (stage 3), the cells of the embryoid body are cultured to select for Central Nervous System (CNS) stem cells. To select for CNS stem cells, the EB cells are plated onto a coated surface that permits adhesion of CNS stem cells, for example a fibronectin-, laminin-, or vitronectin-coated surface. The cells are cultured using a medium which selects for CNS precursor cells, preferably the medium is a serum-free minimal essential medium, such as DMEM or F12, or a combination of DMEM and F12. Preferably, the serum-free medium is supplemented with nutrients such as insulin, selenium chloride, transferrin and fibronectin. An example of a serum free media is ITSFn medium which includes DMEM and F12 in a ratio between 0.1:1 and 10:1 supplemented with between about 1 μg/ml to 10 μg/ml insulin, about 20 nM to about 40 nM selenium chloride, about 40 μg/ml to about 60 μg/ml transferrin and between about 1 μg/ml to 10 μg/ml fibronectin. Generally, the cells are incubated in the serum-free medium for between about 6 to about 8 days at a temperature between about 35° C. and 40° C., preferably about 37° C. under between about 1% and 10% CO₂ atmosphere, more preferably between about 2% and 6% CO₂. Preferably the medium is changed every 1 to 2 days.

According to the invention, at the end of the selection stage, the cell culture contains more than about 50% CNS stem cells, preferably more than about 80% CNS stem cells, more preferably more than about 90% CNS stem cells. The CNS stem cells can be identified by the cell-specific protein nestin Additionally, transcriptional regulators typical of the midbrain and hindbrain such as Pax2, Pax5, Wnt1 and En1 are expressed by the CNS precursor cells.

4. Expansion of the CNS Precursor Cells

In the subsequent step (stage 4), the CNS precursors are expanded for about 6 to about 7 days, preferably until the amount of cells increases about 10 fold to about 100 fold. A variety of culture media are known and are suitable for use in this step. Generally, the proliferation media includes a minimal essential media such as DMEM and/or F12, preferably a combination of DMEM and F12 (at a ratio between about 0.1:1 to 10:1). Preferably the minimal essential media is supplemented with various nutrients such as glucose (between 0.5 mg/ml and 5.0 mg/ml), glutamine (between 0.01 mg/ml and 0.1 mg/ml), sodium bicarbonate (NaHCO₃) (between 0.05 mg/ml and 5.0 mg/ml), insulin (between 10 mg/ml and 30 mg/ml), transferrin (between 50 mg/ml and 150 mg/ml), putrescine (between 50 μM and 150 μM), selenite (between 20 nM and 40 nM) and progesterone (between 10 nM and 30 nM). Preferably, the media includes between about 0.05 mg/ml and 5.0 mg/ml, more preferably between about 1.0 mg/ml to 2.0 mg/ml sodium bicarbonate. Preferably the media does not include 4-(2-hydroxyethyl)-1-piperazine-ethanesulfonic acid (HEPES). A preferred culture media includes N2 media.

The CNS proliferation media may also be supplemented with neurologic agents to encourage differentiation into neuron cells such as secreted signaling factors. Preferably, the culture media includes between about 5 ng/ml to about 30 ng/ml, more preferably between about 10 ng/ml to about 20 ng/ml basic fibroblast growth factor (bFGF) or epidermal growth factor (EGF). Most preferably, between about 10 ng/ml and about 20 ng/ml bFGF is included in the proliferation media.

The culture media may also be supplemented with neurologic agents to increase the efficiency of the generation of midbrain dopaminergic neurons, such as factors that control dopaminergic and serotonergic cell fates during embryogenesis in vivo. Preferably, the media includes about 100 ng/ml to about 1000 ng/ml, more preferably between about 250 ng/ml and 500 ng/ml sonic hedgehog (SHH) protein (or functional fragments thereof) and about 25 ng/ml to about 200 ng/ml, more preferably between about 50 ng/ml to about 100 ng/ml fibroblast growth factor-8 (FGF8) (or functional fragments thereof). Preferably, the media includes both FGF8 and SHH. The inventors discovered that the SHH and FGF8 have a synergistic effect, such that these factors are both significantly less effective when added singly than when added in combination.

The inventors discovered that application of neurologic agents, such as SHH and FGF8, at earlier stages (e.g., stages 1 and 2) was less effective for generating dopaminergic neurons. However, neurologic agents such as SHH and/or FGF8 may be included during stages 1 and 2 if desired.

5. Differentiation of the Expanded CNS Precursors

Differentiation of the expanded CNS precursors to form mature neuronal cells is induced by withdrawal of at least one neurologic agent, typically bFGF (or EGF) (stage 5). Preferably, differentiation is induced by culturing the cells in media similar to the culture media used in stage 4, but without at least one neurologic agent (e.g., bFGF). Additionally, the media may contain factors to enhance dopaminergic neuron yield, such as between about 50 nM to about 500 nM ascorbic acid (AA), more preferably between about 100 nM and 300 nM AA, most preferably between about 150 nM and 250 nM AA. Ascorbic acid treatment tends to increase dopaminergic neuronal population in primary mesencephalic (midbrain) stem cell cultures. (FIG. 4). Typically, when ascorbic acid is added during stage 5 greater than 40% and even greater than 45% of the neurons derived from ES cells express either dopamine or serotonin.

It is noteworthy that, after stage 4, the expanded cell population remains nestin+ (FIG. 2) and therefore retains characteristics of CNS progenitor population.

6. Nurr1-Transfected Cells

In one embodiment, the ES cells are transfected with an exogenous gene encoding a steroid/thyroid hormone nuclear receptor, such as Nurr1, prior to differentiation according to the method described above. Transfecting ES cells with Nurr1 prior to differentiation according to the method described above can increase dopamine expression between 50 to 5000 fold, more typically between 100 and 1000 fold. Additionally, Nurr1-transfected cells can increase the number of dopaminergic neurons generated using the method of the invention 2 to 10 times, more typically 4 to 5 times. While not wishing to be bound by theory, it is believed that Nurr1 interacts with Engrailed (En-1; expressed in midbrain cells) to enhance TH expression.

The increase in TH expression due to transfection with Nurr1 is associated largely with midbrain precursor cells. TH expression does not appear to be increased similarly in serotonin cells.

While Nurr1 appears to be expressed in the transfected stem cells, it does not appear to be expressed in the differentiated neurons. Generally, the undifferentiated Nurr1 transfected ES cells express some TH, indicating that Nurr1 may be activating TH expression prematurely.

i. Vectors

The nucleic acid (e.g., cDNA or genomic DNA) encoding Nurr1 may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, control sequences, such as a signal sequence, an origin of replication, a promoter, a ribozyme binding site, polyadenylation signals, an enhancer element, and/or a transcription termination sequence. The “control sequences” are DNA sequences operably linked to the desired coding sequence. Additionally, a vector may contain one or more marker genes. Construction of suitable vectors containing one or more of these components employs standard ligation techniques that are known to the skilled artisan.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking may be accomplished by ligation at convenient restriction sites. If such sites do not exist, the synthetic oligonucleotide adaptors or linkers may be used in accordance with conventional practice.

Typical selection markers encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli. Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

Promoters recognized by a variety of potential host cells are well known. Promoters suitable for use with in mammalian cells include promoters obtained from the genomes of viruses such as polyoma virus, fowlpox virus (UK 2,211,504 published Jul. 5, 1989), adenovirus (such as Adenovirus 2), bovine papilloma virus, avian sarcoma virus, cytomegalovirus, a retrovirus, hepatitis-B virus, and Simian Virus 40 (SV40), from heterologous mammalian promoters, e.g., the actin promoter or an immunoglobulin promoter, and from heat-shock promoters, provided such promoters are compatible with the host cell systems.

Enhancer sequences are typically cis-acting elements of DNA, usually about from 10 to 300 bp, that act on apromoter to increase its transcription. Many enhancer sequences from mammalian genes are known (globin, elastase, albumin, α-fetoprotein, and insulin).

Termination sequences are also known. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the gene.

ii. Transfection Methods

Methods of transfection are known to the ordinarily skilled artisan and include, for example, lipofectin, CaPO₄, and electroporation. Depending on the host cell used, transformation is performed using standard techniques appropriate to such cells. For mammalian cells without such cell walls, the calcium phosphate precipitation method of Graham and van der Eb, Virology, 52:456-457 (1978) can be employed. General aspects of mammalian cell host system transformations have been described in U.S. Pat. No. 4,399,216. For various techniques for transforming mammalian cells, see Keown et al., Methods in Enzymology, 185:527-537 (1990) and Mansour et al., Nature, 336:348-352 (1988).

iii. Gene Expression

Gene expression may be measured in a sample directly, for example, by conventional Southern blotting, Northern blotting to quantitate the transcription of mRNA (Thomas, Proc. Natl. Acad. Sci. USA, 77: 5201-5205 (1980)), dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal.

E. Differentiated Cell Culture

Another aspect of the invention is directed to a cell culture which includes at least about 50%, more preferably at least about 70%, most preferably at least about 80% neurons. Of the neurons in the cell culture, typically between at least about 20%, more typically at least about 30%, and most preferably at least about 40% are TH+ cells such as dopaminergic neurons. The cell culture also includes glial cells, typically between about 1% to about 3% astrocytes. At least some of the differentiated neurons in the cell culture are synaptically active. Typically, at least about 90%, more typically at least about 95% of the cells in the culture are synaptically active differentiated neurons (e.g., dopaminergic and/or serotonergic neurons).

Another aspect of the invention provides a cell culture that includes cells transfected with Nurr1. Typically, the differentiated transfected cell culture includes at least about 50%, more preferably at least about 65%, most preferably at least about 95% neurons. Of the neurons in the cell culture, typically between at least about 5%, more typically at least about 25%, and most preferably at least about 80% are TH+ cells such as dopaminergic neurons. The cell culture also includes glial cells, typically between about 5% to about 50% astrocytes. At least some of the differentiated neurons in the cell culture are synaptically active. Typically, at least about 10%, more typically at least about 80% of the cells in the culture are synaptically active differentiated neurons (e.g., dopaminergic and/or serotonergic neurons). The transfected cells typically release about 1 ng/ml to about 500 ng/ml dopamine per cell, more typically about 10 ng/ml to about 200 ng/ml, most typically about 100 ng/ml to about 150 ng/ml dopamine per cell, for example, by KCl-evoked dopamine release.

F. Disorders of the Central Nervous System

The present method can be employed to deliver agents to the brain for diagnosis, treatment or prevention of disorders or diseases of the CNS, brain, and/or spinal cord. These disorders can be neurologic or psychiatric disorders. These disorders or diseases include brain diseases such as Alzheimer's disease, Parkinson's disease, Lewy body dementia, multiple sclerosis, epilepsy, cerebellar ataxia, progressive supranuclear palsy, amyotrophic lateral sclerosis, affective disorders, anxiety disorders, obsessive compulsive disorders, personality disorders, attention deficit disorder, attention deficit hyperactivity disorder, Tourette Syndrome, Tay Sachs, Nieman Pick, and other lipid storage and genetic brain diseases and/or schizophrenia. The method can also be employed in subjects suffering from or at risk for nerve damage from cerebrovascular disorders such as stroke in the brain or spinal cord, from CNS infections including meningitis and HIV, from tumors of the brain and spinal cord, or from a prion disease. The method can also be employed to deliver agents to counter CNS disorders resulting from ordinary aging (e.g., anosmia or loss of the general chemical sense), brain injury, or spinal cord injury.

The present method can be employed to deliver agents to the brain for diagnosis, treatment or prevention of neurodegenerative disorders. Sublingual, conjunctival or transdermal administration of an agent to peripheral nerve cells of the trigeminal and other sensory neural pathways innervating the skin or the conjunctival or oral mucosa, purported entryway for causative agents of brain diseases, can help protect against disease in these nerve cells and regenerate injured nerve cells, thereby forestalling the subsequent spread of disease to susceptible areas of the CNS, brain, and/or spinal cord.

The application of an agent to the sublingual, conjunctival, or facial epithelium can also help prevent the spread of certain CNS, brain, and/or spinal cord disorders by directly treating peripheral cells and neurons that are injured by neurotoxins and other insults. Prophylactic treatment of these outlying nerve cells helps preclude the entrance of disease-causing agents into the CNS, brain, and/or spinal cord. This method of treatment is particularly beneficial in cases of Alzheimer's disease where an environmental factor is suspected of being one of the causative agents of the disease. Application of an agent to the sensory neurons also in part treats or prevents the loss of smell or of the general chemical sense which may be associated with neurodegenerative diseases and ordinary aging.

Treatment of Parkinson's disease may also be an important application of the present delivery method since the trigeminal nerve pathway can deliver neurotrophins and other therapeutic agents from the oral cavity, conjunctiva, or skin to the pons in the brain stem. The principal therapeutic target in the brain for Parkinson's is the substantia nigra which extends forward over the dorsal surface of the basis peduncle from the rostral border of the pons toward the subthalamic nucleus. Other therapeutic target areas are the locus ceruleus which is located in the rostral pons region and the ventral tegmental area which is located dorsomedial to the substantia nigra.

The method of the present invention may be used with any mammal. Exemplary mammals include, but are not limited to rats, cats, dogs, horses, cows, sheep, pigs, and more preferably humans.

G. Methods of Use

In another embodiment, the invention provides a method of treating a patient suffering from a neurological disorder, such as a central nervous system disorder, or alleviating the symptoms of such a disorder, by administering cells cultured according to the method of the invention to the patient's brain. Examples of neurological disorders include Parkinson's disease, Huntington's disease, Alzheimer's disease, severe seizure disorders including epilepsy, familial dysautonomia as well as injury or trauma to the nervous system, such as neurotoxic injury or disorders of mood and behavior such as addiction and schizophrenia.

In this method of the invention, cells are cultured as described above to form differentiated neuronal cells which are then transplanted into the brain of a patient in need thereof.

1. Formulations

After the differentiated neuronal cells are formed according to the cell culturing method previously described, the cells are suspended in a physiologically compatible carrier. As used herein, the term “physiologically compatible carrier” refers to a carrier that is compatible with the other ingredients of the formulation and not deleterious to the recipient thereof. Those of skill in the art are familiar with physiologically compatible carriers. Examples of suitable carriers include cell culture medium (e.g., Eagle's minimal essential media), phosphate buffered saline, and Hank's balanced salt solution +/− glucose (HBSS).

The volume of cell suspension administered to a patient will vary depending on the site of implantation, treatment goal and amount of cells in solution. Typically the amount of cells administered to a patient will be a “therapeutically effective amount.” As used herein, a therapeutically effective amount refers to the number of transplanted cells which are required to effect treatment of the particular disorder. For example, where the treatment is for Parkinson's disease, transplantation of a therapeutically effective amount of cells will typically produce a reduction in the amount and/or severity of the symptoms associated with that disorder, e.g., rigidity, akinesia and gait disorder.

It is estimated that a severe Parkinson's patient will need at least about 100,000 surviving dopamine cells per grafted side to have a substantial beneficial effect from the transplantation. As cell survival is low in brain tissue transplantation in general (5-10%) an estimated 1-4 million dopaminergic neurons should be transplanted.

2. Methods of administration

According to the invention, the cells are administered to the patient's brain. The cells may be implanted within the parenchyma of the brain, in the space containing cerebrospinal fluids, such as the sub-arachnoid space or ventricles, or extaneurally. As used herein, the term “extraneurally” is intended to indicate regions of the patient which are not within the central nervous system or peripheral nervous system, such as the celiac ganglion or sciatic nerve. “Central nervous system” is meant to include all structures within the dura mater.

Typically, the neuronal cells are administered by injection into the brain of the patient. Injections can generally be made with a sterilized syringe having an 18-21 gauge needle. Although the exact size needle will depend on the species being treated, the needle should not be bigger than 1 mm diameter in any species. Those of skill in the art are familiar with techniques for administering cells to the brain of a patient.

3. Diseases

a. Parkinson's Disease

Parkinson's disease (PD) is characterized by the progressive loss in function of dopaminergic neurons. The progressive loss of dopaminergic function interferes with the normal working of the neuronal circuitry necessary for motor control so that patients with PD show characteristic motor disturbances such as akinesia, rigidity and rest tremor. Other symptoms include pain, impaired olfaction, alterations of personality and depression. Quinn et al., (1997) Baillieres Clin. Neurol. 6:1-13.

According to the invention, dopaminergic neuronal cells are generated using the cell culturing method described above. The dopaminergic cells are then administered to the brain of the patient -in need thereof to produce dopamine and restore behavioral deficits in the patient. Preferably, the cells are administered to the basal ganglia of the patient.

b. Alzheimer's disease

Alzheimer's disease involves a deficit in cholinergic cells in the nucleus basalis. Thus, a subject having Alzheimer's disease may be treated by administering cells cultured according to the method of the invention that are capable of producing acetylcholine.

c. Huntington's Disease

Huntington's disease involves a gross wasting of the head of the caudate nucleus and putamen, usually accompanied by moderate disease of the gyrus. A subject suffering from Huntington's disease can be treated by implanting cells cultured according to the method of the invention that are capable of producing the neurotransmitters gamma amino butyric acid (GABA), acetylcholine, or a mixture thereof.

4. Gene Therapy

In an additional embodiment of the invention, the cultured cells may be transfected with a nucleic acid which encodes a neurologically relevant polypeptide. The term “neurologically relevant peptide” generally refers to a peptide or protein which catalyzes a reaction within the tissues of the central nervous system. Such peptides may be naturally occurring neural peptides, proteins or enzymes, or may be peptide or protein fragments which have therapeutic activity within the central nervous system.

According to this aspect of the invention, precursor cells are cultured in vitro as described above and an exogenous gene encoding a desired gene product is introduced into the cells, for example, by transfection. The transfected cultured cells can then be administered to a patient with a neurological disorder.

a. Genes of Interest

Examples of neurologically relevant peptides include neural growth factors and enzymes used to catalyze the production of important neurochemicals or their intermediates. The peptide encoded by the nucleic acid may be exogenous to the host or endogenous. For example, an endogenous gene that supplements or replaces deficient production of a peptide by the tissue of the host wherein such deficiency is a cause of the symptoms of a particular disorder. In this case, the cell lines act as an artificial source of the peptide. Alternatively, the peptide may be an enzyme which catalyzes the production of a therapeutic or neurologically relevant compound. Again, such compounds may be exogenous to the patient's system or may be an endogenous compound whose synthetic pathway is otherwise impaired. Examples of neurologically relevant compounds include tyrosine hydroxylase, nerve growth factor (NGF), brain derived neurotrophic factor (BDNF), basic fibroblast growth factor (bFGF) and glial cell line derived neurotrophic factor (GDNF).

b. Gene Constructs

Typically the gene of interest is cloned into an expression vector. As used herein, the term “expression vector” refers to a vector which (due to the presence of appropriate transcriptional and/or translational control sequences) is capable of expressing a DNA molecule which has been cloned into the vector and of thereby producing a polypeptide or protein. A nucleic acid molecule, such as DNA, is said to be “capable of expressing” a polypeptide if it contains nucleotide sequences which contain transcriptional and translational regulatory information and such sequences are “operably linked” to a nucleotide sequence that encodes the polypeptide. An operable linkage is a linkage in which the regulatory DNA sequences and the DNA sequence sought to be expressed are connected in such a way as to permit gene expression. Regulatory elements include elements such as a promoter, an initiation codon, a stop codon and a polyadenylation signal.

Expression of the cloned sequences occurs when the expression vector is introduced into an appropriate host cell. In this case, the preferred host cell is a ES Cell that, upon differentiation, generates neuronal cell. Procedures for preparing expression vectors are known to those of skill in the art and can be found in Sambrook et al., Molecular Cloning: A Laboratory Manual, 2nd Ed., Cold Spring Harbor Press, Cold Spring Harbor, N.Y. (1989).

5. Genetically Modified Cells

ES cells are receptive to genetic modification. Thus, ES cells can be genetically modified before transplantation to enhance therapy. For example, the cells can be genetically engineered to decrease rejection, for example, the immunogenicity of the cell may be suppressed by deleting genes that produce proteins that are recognized as “foreign” by the host, or by introducing genes which produce proteins, such as proteins that are native to the host and recognized as “self” proteins by the host immune system. Alternately, the ES cells can be engineered to increase survival by introducing genes that produce growth factors that promote survival of neural cells, such as neurotrophins NT-3, NT-4/5, BDNF and NGF.

H. Assay

Another aspect of the invention provides an assay for evaluating the effect of substances on differentiated cells, preferably differentiated neuronal cells. The assay can be used to develop drugs capable of regulating the survival, proliferation or genesis of neuronal cells. The assay can also be used to screen for antagonists and/or agonists of dopamine or serotonin. According to this aspect of the invention, a population of neuronal cells is produced by the cell culturing method described above. The population of cells is contacted with a substance of interest and the effect on the cell population is monitored. The impact on the cell population can be monitored, for example, by determining whether the substance causes an increase or decrease in the expression of a reporter gene by examining the level of its protein, RNA, biological activity or other methods. For example, in one immunocytochemical method, the dopaminergic cells are monitored to determine the impact of a substance on the expression of tyrosine hydroxylase.

Substances of interest include extracts from tissues or cells, conditioned media from primary cells or cell lines, polypeptides whether naturally occurring or recombinant, nucleotides (DNA or RNA) and non-protein molecules whether naturally occurring or chemically synthesized.

The cell culture can also be used to studying the mechanism of neurotransmitter synthesis and release, particularly for serotonin and dopamine, neuron cell survival, and the elecrophysiochemical properties of differentiated neuronal cells (such as serotonergic and dopaminergic cells).

The cell culture can also be used to evaluate the role of various genes in differentiated neuron cells. For example, a specific gene (for example, the patched receptor gene for SHH) may be knocked out in an ES cell. Methods for producing knock out variants are known. The knocked out ES cell (for example, a homozygous null mutant) can be cultured to form differentiated neuronal cells according to the above-described method. The culture of differentiated neuronal cells can then be used to examine the function of the knocked out gene.

### WORKING EXAMPLES

### Example 1

### Culturing Embryonic Stem (ES) Cells.

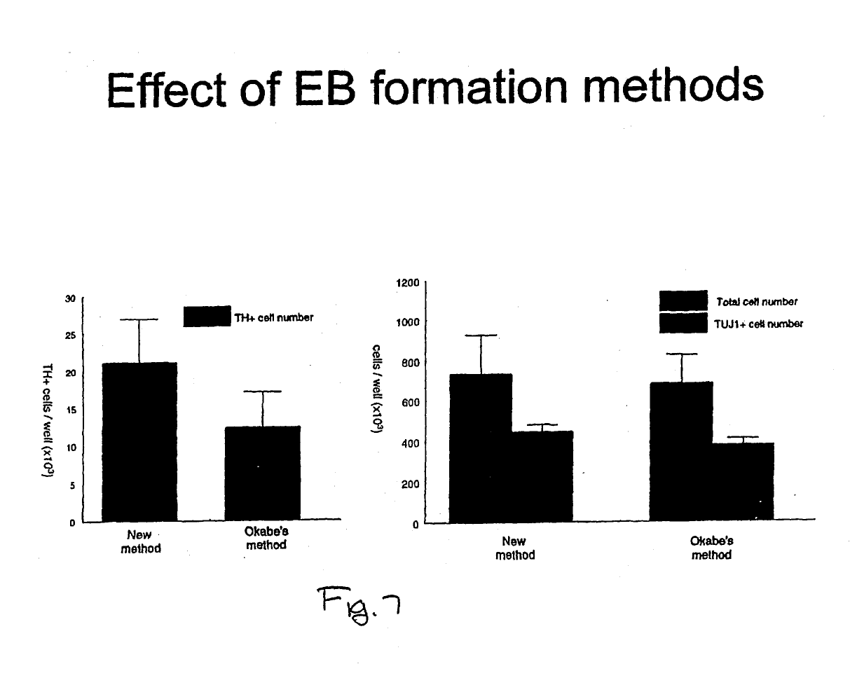

The present invention provides a method for generating midbrain neurons from ES cells. The method of the invention is a modification of the method described by Okabe et al., (1996) “Development of neuronal precursor cells and functional postmitotic neurons from embryonic stem cells in vitro,” Mech. Dev. 59:89-102. The modifications result in the production of dopaminergic neurons rather than GABAergic and glutamertgic neurons. FIG. 7 compares the amount of TH+ cells and TUJ1+cells obtained by the method of the invention compared to the method of Okabe et al.

Undifferentiated embryonic stem (ES) cells (R1, E14.1 and B5, obtained from Dr. Heimer Westphal, NIH, Bethesda, Md., and Dr. Tom Doetschman, University of Cincinnati, Cincinnati, Ohio, were cultured essentially as described by Okabe et al., (1996) “Development of neuronal precursor cells and functional postmitotic neurons from embryonic stem cells in vitro,” Mech. Dev. 59:89-102, with some modifications.

Briefly, undifferentiated ES cells were grown for 6 days at 37° C. under 5% CO₂ on gelatin coated tissue culture plates (0.1% gelatin, commercially available from Sigma under the brand name Falcon) and in the presence of 1400 units/ml of leukemia inhibitory factor (LIF, GIBCO/BRL, Grand Island N.Y.). The medium was changed daily.

The LIF was prepared by combining 1.4 ml of a stock solution with 10 ml medium and was included to prevent differentiation.

100 ml ES cell medium was prepared by combining 82 ml knock out DMEM medium (GIBCO/BRL) supplemented with 15 ml (15%) fetal calf serum (FCS; ES cell qualified; Gibco BRL 16141-661)), 1 ml (100 μM) minimal essential medium (MEM; Gibco BRL 11140-050), 0.1 ml (0.5 μM) 2-mercaptoethanol (Gibco BRL 21985-023), 1 ml L-glutamine (Gibco BRL), and 1 ml antibiotics (streptomycin and penicillin).

### Example 2

### Embryoid Bodies Formation

In the method by Okabe et al., EB formation was initiated from clusters of undifferentiated ES cells without dissociating them to form a population of single cells. In contrast, in this example, the clusters of undifferentiated ES cells are dissociated into a population of single cells. The EB are then formed from the individual cells.

Briefly, to induce EB formation (stage 2), the cells from stage 1 were dissociated into single cells by adding a solution containing 0.05% trypsin and 0.04% ethylene diaminetetraacetate (EDTA) in phosphate buffered saline (PBS) to the culture dishes for 5 minutes. After the cells were dissociated, they were plated, using a pipet, onto non-adherent bacterial culture dishes (commercially available from Falcon) at a density of 1.5-2×10⁶ cells/10 cm² dish and incubated for 4 days in the ES media described above. The medium was changed every 2 days.

When the incubation was complete, the cells were transferred to 15 ml test tube and the cells were allowed to settled to the base of the tube. Once the cells settled, the medium was removed and replaced with fresh ES medium. The cells were then transferred to tissue culture plates and incubated for 24 hours (overnight) at 37° C. in 5% CO₂ incubator.

Forming the EB from individual ES cells results in a 3.5 fold increase in the yield of nestin positive cells. (6.4% nestin+ cells/ES cells were generated with this procedure). Moreover, the method in this example generates more tyrosine hydroxylate+ (TH, a marker for dopaminergic neurons) neurons and TUJ1+ (general marker for neurons) neurons per nestin+ cell after stage 5. Finally, 6.0 fold and 4.2 fold more TH+ and TUJ1+ neurons per undifferentiated cell, respectively, were generated by this method.

### Example 3

### Cell Selection Process: Selection of Nestin+ Cells

After the cells were cultured for 24 hours, selection of nestin+ cells was initiated. Briefly, selection of nestin+ cells (stage 3) was initiated by replacing the ES cell medium by serum-free ITSFn medium. The cells were then incubated in the ITSFn medium for 6-10 days, changing the medium every 2 days.

The ITSFn medium contained DMEM and F12 (commercially available from Life Tech) in a 1:1 ratio; 5 μg/ml insulin 30 nM selenium chloride; 50 μg/ml transferrin; 5 μg/ml fibronectin. The fibronectin was added separately when the medium was changed. The fibronectin solution was prepared by combining 50 μl of 1 mg/ml stock with 10 ml medium.

### Example 4

### Expansion of Nestin+ Cells

After 6 days of selection, cell expansion (stage 4) was initiated, using a modification of Okabe et al., using N2 medium instead of N3FL medium.

Specifically, the cells were dissociated by adding a solution of 0.05% typsin and 0.04% EDTA to the plates and incubating at 37° C. for 5 minutes. The dissociated cells were plated on tissue culture plastic or glass coverslips at a concentration of 1.5-2×10⁶ cm² in serum free N2 medium supplemented with 1 μg/ml of laminin and 10 ng/ml of basic fibroblast growth factor (bFGF, R&D Systems, Minneapolis, Minn.). The medium was also supplemented with murine aminoterminal fragment SHH (500 ng/ml); murine FGF8 isoform b (100 ng/ml, both from R& D System); and ascorbic acid (200 μM, Sigma). Control cultures were treated identically except that no SHH or FGF8 was applied during cell proliferation.

The N2 medium was prepared according to Bottenstein et al., (1979) “Growth of a rat neuroblastoma cell line in serum-free supplemented medium,” Proc. Natl. Acad. Sci. U.S.A. 76:514-517 with the modifications of Johe et al., (1996) “Single factors direct the differentiation of stem cells from the fetal and adult central nervous system,” Genes and Development 10:3129-3140.

Prior to cell plating, dishes and coverslips were precoated with polyornithine (15 μg/ml) and laminin (1 μg/ml, both from Becton Dickinson Labware, Bedford, Mass.).

The cells were proliferated for 6-7 days and the medium was changed every two days.

### Example 5

### Generation of Dopaminergic Neurons: Differentiation to TH+ Neuron

Differentiation (stage 5) was induced using a modification of Okabe et al., using N2 medium instead of N3FL medium. Differentiation was induced by withdrawing the mitogen bFGF from the medium and maintaining the cells serum-free N2 medium supplemented with laminin (1 μg/ml) for 5-6 days (stage 5 cells, FIG. 1) (i.e., the differentiation medium contained no bFGF, no SHH, and no FGF8 but was supplemented with 1 μg/ml laminin).

Combined treatment with SHH/FGF8 during stage 4 (expansion, FIG. 1) resulted in about a 2.3 fold increase in the number of TH+ cells (15.4±2.4% of the TUJ1+ neurons; n>40, p<0.001). However, the same treatment during stage 5 (differentiation) of culture proved ineffective (FIG. 4)

### Example 6

### Comparison of N2 and N3FL Media

The use of N2 medium resulted in a 4.8 and 13.2 fold increase in the number of total cells and TUJ1+ neuronal cells, respectively, in culture after 6 days of differentiation. While 4.1% of TUJ1+ neurons were TH+ in cultures grown in N2 medium, no TH+ cells were detected in cultures grown in N3FL media.

Since the composition of N2 and N3FL media is relatively similar (Table 1, below), this experiment was performed to determine which of the media component(s) most influence the composition of the resulting cell population.

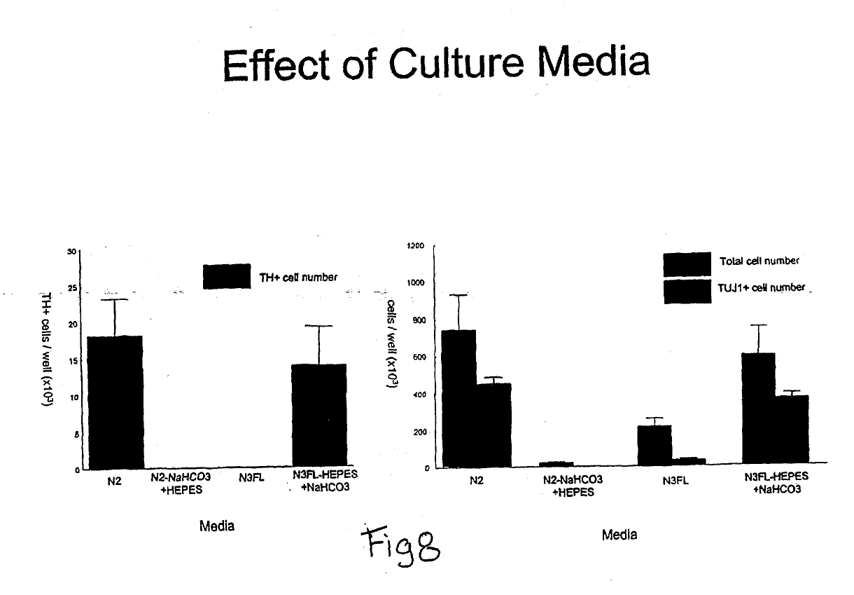

Variation of transferrin, glucose and glutamine concentrations in the medium did not significantly affect the phenotype of the ES cell derived population, however, substitution of bicarbonate (N2 medium) by HEPES (N3FL) medium dramatically reduced the percentage of TUJ1 and TH+ cells. See FIG. 8. Thus, we conclude that HEPES present in N3FL medium reduces the amount of TH+ cells in the culture.

TABLE 1
Composition of N2 and N3FL media (per 500 ml)

| | N2 | | N3FL | |
|---|---|---|---|---|
| DMEM/F12 | 6 | g | 6 | g |
| Glucose | 0.775 | g | — | |
| Glutamine | 0.0365 | g | — | |
| Insulin | 12.5 | mg | 12.5 | mg |
| Transferrin | 50 | mg | 25 | mg |
| Puteresine | 100 | μM | 100 | μM |
| Selenite | 30 | nM | 30 | nM |
| Progesterone | 20 | nM | 20 | nM |
| NaHCO₃ | 0.845 | g | — | |
| HEPES | — | | 1.95 | g |
| Streptomycin/ | 5 | ml | 5 | ml |
| Penicillin | | | | |

Using N2 media, the percentage of TUJ1+ cells was 71.9±6.9% of the total cell population. Without growth factor treatment (SHH and FGF8 during stage 4 and AA during stage 5) 6.9±1.5% of all TUJ1 cells were TH+. Treatment of ES cells with the growth factors dramatically increased the percentage of TH+ cells to 33.9±5.5%.

### Example 7

### Immunohistochemical Identification (TH or TUJI)

The cell morphology and phenotype of the cells from Example 6 were analyzed using immunocytochemistry. Although TH immunoreactivity is commonly used for the identification of midbrain dopaminergic neurons, TH is also expressed in other cells, such as peripheral nervous system (PNS) neurons, noradrenergic neurons, adrenergic neurons, and striatal progenitors that transiently co-express TH in inhibitory GABAergic cells during development. Therefore, to further characterize the ES derived cell population, double immunohistochemistry analysis was performed for TH/dopamine-β hydroxylase (DBH), a marker of noradrenergic neurons and for TH/GABA and TH/Serotonin. No coexpression of TH with any of these markers was observed suggesting that the cells are dopaminergic neurons. Furthermore, the ES derived cells exhibited typical morphological characteristics of catecholaminergic neurons: the were mostly bipolar in shape with numerous varicosities along the neurites and often arranged in clusters of TH cells.

To perform the immunohistochemical analysis, the following primary antibodies were used (dilutions were performed using dilution with phosphate buffered saline (PBS) containing 0.1% bovine serum albumin (BSA) and 10% Normal Goat Serum (NGS)): tyrosine hydroxylase (TH) polyclonal antibody, diluted 1:200 to 1:500 (P40101, Pel-Freeze, Rogers, AR) or TH monoclonal diluted 1:1000 (Sigma); β-tubulin type III (TUJ1) monoclonal, diluted 1:500 (MMS-435P, Babco, Richmond, Calif.); gamma-aminobutryic acid (GABA) polyclonal 1:100 (Sigma, St. Louis, Mo.); dopamine-β-hydroxylase (DBH) 1:100 (Protos Biotech Corp, NY, N.Y.) or DBH 1:100 (Pharmingen, San Diego, Calif.); and serotonin polyclonal 1:2000 (Sigma, St. Louis, Mo.).

For detection of primary antibodies, the following fluorescently labeled secondary antibodies were used for TH staining and TUJ1 staining: biotinylated anti-rabbit IgG, 1:200 dilution (Vector, goat); and biotinylated anti-mouse IgG, 1:200 dilution (Vector, goat), respectively, according to the methods recommended by the manufacturer.

Immunocytochemistry was carried out utilizing standard protocols. Briefly, the cells were fixed for 20 min in phosphate buffered saline (PBS) containing 4% paraformaldehyde and 0.15% picric acid and then washed three times, for 5 minutes each wash, in phosphate buffered saline (PBS) containing 0.1% bovine serum albumin (BSA). After the third wash, the cells were combined with a blocking solution. The blocking solution contained PBS, 0.1% BSA, 10% normal goat serum (NGS) and 0.3% triton X-100. After blocking, primary antibody was applied and the cells were incubated in a cold room (4° C.) overnight, shaking at a speed of about 10-20 rpm. The cells were then washed again, three times, for five minutes each wash, in the same PBS/BSA solution described above. After the third wash, the secondary antibody was applied and the cells were incubated at room temperature for 45 minutes. After incubation with the secondary antibodies, the cells were washed three times, as described above, and a peroxidase solution (Vectastain® ABC kit) was applied. After a 45 minute incubation, the cells were washed three times and diaminobenzidine (DAB) solution was applied for 3-5 min.

Immunohistochemical analyses of the differentiated cultures revealed the presence of large numbers of neurons displaying β-tubulin III (TUJ1) in both control and SHH/FGF/KCl treated cells (data not shown). However, there was a dramatic difference between treated and control cultures in the percentage of specific neurons expressing tyrosine-hydroxylase (TH). The percentage of neurons expressing TUJ1 was 71.9%±6.0. The percentage of TUJI cells expressing TH was 6.9%±1.5% (these numbers are the average of 3 independent experiments, n>40).

Uniform random sampling procedure were used for cell counts and quantified using the fractionator technique (Gundersen et al., (1988) “Some new, simple and efficient stereological methods and their use in pathological research and diagnosis,” APIMS 96:379-394). Statistical comparisons were made by ANOVA with posthoc Dunnett test when more than 2 groups were involved. If data were not normally distributed, a non-parametric test (Mann-Whitney U) was used for the comparison of results. Data expressed as mean±SEM.

As shown in FIG. 4, SHH, FGF8, cAMP, and ascorbic acid increase the yield of TH+ neurons in ES cell cultures. Yield of TH+ neurons is expressed as a percentage of all TUJ1+ neurons. SHH (500 ng/ml), FGF8 (100 ng/ml), cAMP (1 mM), and ascorbic acid (200 μM) were added at different stages of ES cell development, as shown; and (C) combined treatment with SHH, FGFS and ascorbic acid results in 5 fold increase in the number of TH+ cells over the untreated controls. Fluorescence staining was carried out with a TH polyclonal antibody and cyanine Cy2-labeled secondary antibody.

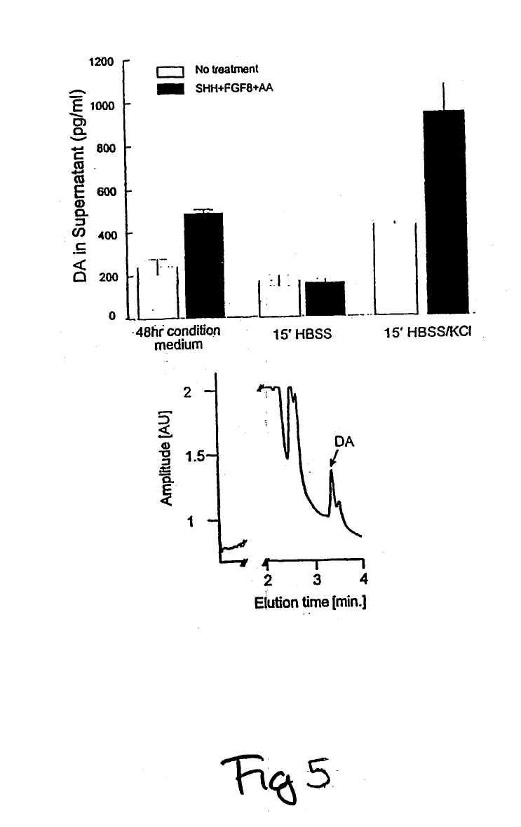

As shown in FIG. 5, TH+ cells do not co-express DBH, GABA, and serotonin. Double staining was carried out with TH (polyclonal), DBH (monoclonal), GABA (monoclonal), and serotonin (monoclonal) primary antibodies, and cyanine Cy3 (TH) and cyanine Cy2 (DBH, GABA, serotonin)-labeled secondary antibodies; Treatment with SHH, FGF8 and ascorbic acid greatly promotes maturation of dopaminergic neurons as measured by increase in KCl-evoked dopamine release. The rpHPLC determination of dopamine concentration in 48 h conditioned N2 medium, in physiological solution (BBSS; 15 min), and in HBSS+56 mM KCl (15 min) is shown. The lower panel shows a typical chromatogram for dopamine elution from the rpHPLC.

### Example 8

### Reverse Phase High Performance Liquid Chromatography

Another measure of dopamine neuron identity is the production of dopamine. Reverse phase high performance liquid chromatography (RP-HPLC) was used to determine whether the ES derived cells secrete significant levels of dopamine.

Dopamine levels in the culture media of the differentiated ES cells were determined after 6 days of differentiation in the conditioned medium (48 hours after medium change), in HBSS (basal release, 15 min. incubation), and in HBSS containing 56 mM KCl (evoked release, 15 min incubation). Culture supernatants, HBSS and HBSS +56 mM KCl were collected, immediately stabilized with orthophosphoric acid (7.5%)/metabisulfate (0.22 mg/ml), and stored at −80° C. until analysis.

Aluminum absorption, equipment and HPLC analysis of dopamine have been described previously (Studer et al., (1998) “Transplantation of expanded mesencephalic precursors leads to recovery in parkinsonian rats,” Nature Neurosci. 1:290-295; and Studer et al., (1996) “Non-invasive dopamine determination by reversed phase HPLC in the medium of free-floating roller tube cultures of rat fetal ventral mesencephalon. A tool to assess dopaminergic tissue prior to grafting,” Brain Res.Bull. 41:143-150). Results were normalized against dopamine standards at varying flow rates and sensitivities.

When the control medium was subsequently evaluated by RP-HPLC, 231.8±34.2 pg/ml of dopamine was present. The dopamine levels of the medium treated with HBSS measured 165.7±23.4 pg/ml (the growth factor treated culture was not significantly different). The dopamine level in the cell culture treated with SHH/FGF8/AA was over 2 fold higher (n=3, p<0.05). See FIG. 5.

In the control cultures, 416.6±72.pg/ml dopamine was released. In the growth factor treated cultures the dopamine level increased 6-fold over the appropriate control to 918.4±123.2 pg/ml It is believed that treating the culture was with elevated potassium for 15 min causes an increase in dopamine release due to depolarization of the cellular membrane.

### Example 9

### Generation of Serotonergic Neurons

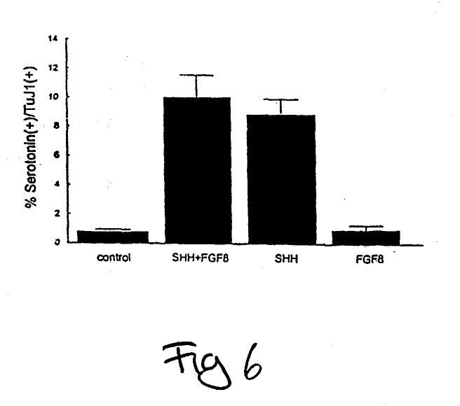

In addition to its role in the differentiation of dopaminergic neurons, SHH is also important for the generation of hindbrain serotenergic neurons. To test whether growth factors SHH and FGF8 increase the yield of serotenergic neurons in an ES culture system, control and SHH/FGF8 treated cultures were subjected to double immunohistochemistry for serotonin and TH (FIG. 6) using a method similar to that described in Example 7 using TH monoclonal antibody (Sigma) and serotonin polyclonal antibody (Sigma).

In the untreated cultures (i.e., no SHH or FGF8), 0.8±0.1% (n>40) of all TUJ1 neurons stained positive for serotonin. In cultures treated with SHH/FGF8 during stage 4 (expansion) the serogenergic population increased 14-fold (11.0±0.5%, n>40, p<0.01 of all TJU1+ cells).

Interestingly, application of SHH in the absence of exogenous FGF8 promoted serotonergic fates to an extent similar to that of the combined treatment (SHH and FGF8) (FIG. 6). These results suggest that FGF8 might be required for specification of some, but not all the types of serotonergic neurons. The efficient induction of serotonergic neurons suggests that the differentiation conditions developed to promote midbrain dopaminergic fates also promote hindbrain serotonergic fates. Our findings suggest that almost half of the neurons can adopt a ventral mid/hindbrain fate under this protocol.

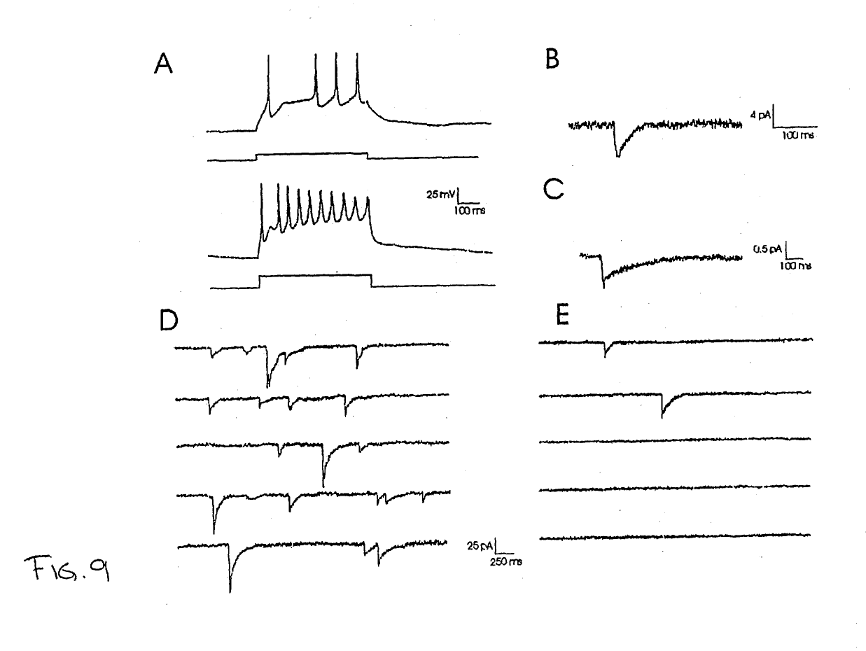

FIG. 9 is a bar graph showing that SHH and FGF8 promote generation of serotonergic neurons. (A) SHH (500 ng/ml), and FGF8 (100 ng/ml) increase the yield of serotonergic neurons over the untreated controls. TH and serotonin are not co-expressed by the same cells; and (B) SHH alone promotes serotonergic fate to the extent similar to that of the combined treatment. Yield of serotonin+ neurons is expressed as a percentage of all TUJ1+ neurons.

### Example 10

### Protein Expression

The developmental progression of ES cells was investigated by examining the appearance of CNS and dopaminergic specific regulatory gene products (using RT-PCR) at different stages of ES cell development.

To analyze relative expression of different mRNAs, the amount of cDNA was normalized based on the signal from ubiquitously expressed actin mRNA. Levels of neural mRNAs at different stages of ES cell culture was compared to that in the undifferentiated ES cells.

A. Cell Isolation

Cells were isolated from the culture on the last day of the various stages (stages 1-5 in FIG. 1) of the process described in Examples 1-6. Cell isolation methods are known to those of skill in the art and may vary depending on the stage.

B. Cell Preparation

After the cells were isolated, the cells were prepared using known protocols. The precise method varied depending on the stage. For example, EBs (stage 2) were harvested by centrifugation and then lysed for RNA. The cells from stages 3, 4 and 5 were lysed directly on the tissue culture plates.

C. RNA Preparation

Total cellular RNA was prepared using RNAeasy total RNA purification kit (Qiagen, Valencia, Calif.) following the manufacturer's instructions. The total cellular RNA was then treated with RNase-free RQ DNase (Promega Corp., Madison, Wis.) to remove traces of DNA.

D. cDNA Preparation

The cDNA synthesis was carried out using Moloney murine leukemia virus (M-MLV) Superscript II reverse transcriptase (GIBCO/BRL) following the manufacturer's instructions. Random hexamer primers (GIBCO/BRL) were used to prime reverse transcriptase (RT) reactions. Using this method it was possible to use the same RT reaction (cDNA) for PCR amplification with different set of gene-specific primers.

E. PCR Amplification

The PCR was carried out using standard protocols with Taq polymerase (Boehringer-Mannheim, Indianapolis, Ind.). Cycling parameters were as follows: denaturation at 94° C. for 30 sec, annealing at 58-61° C. for 1 minute depending on the primer, and elongation at 72° C. for 1 minute. The number of cycles varied between 25 and 35, depending on the particular mRNA abundance. The number of cycles and the amount of cDNA was chosen in such a way as to select PCR conditions on the linear portion of the reaction curve avoiding “saturation effects” of PCR. Primer sequences and the length of the amplified products were as follows (forward primer is shown on top; and reverse primer is shown on bottom):

| | | |
|---|---|---|
| actin (569): | ATG GAT GAC GAT ATC GCT G | (SEQ. ID. NO: 1) |
| | ATG AGG TAG TCT GTC AGG T | (SEQ. ID. NO: 2) |
| Nestin(327): | GGA GTG TCG CTT AGA GGT GC | (SEQ. ID. NO: 3) |
| | TCC AGA AAG CCA AGA GAA GC | (SEQ. ID. NO: 4) |
| Nurr1(255): | TGA AGA GAG CGG AGA AGG AGA TC | (SEQ. ID. NO: 5) |
| | TCT GGA GTT AAG AAA TCG GAG CTG | (SEQ. ID. NO: 6) |
| Gli1(462): | TCC ACA GGC ATA CAG GAT CA | (SEQ. ID. NO: 7) |
| | TGC AAC CTT CTT GCT CAC AC | (SEQ. ID. NO: 8) |
| Smo(370): | CTG AGA GTG CCA GAA AAG GG | (SEQ. ID. NO: 9) |
| | TCA TCA TGC TGG AGA ACT CG | (SEQ. ID. NO: 10) |
| Ptc(272): | CCT CCT TTA CGG TGG ACA AA | (SEQ. ID. NO: 11) |
| | ATC AAC TCC TCC TGC CAA TG | (SEQ. ID. NO: 12) |
| Wnt1(462): | ACC TGT TGA CGG ATT CCA AG | (SEQ. ID. NO: 13) |
| | TCA TGA GGA AGC GTA GGT CC | (SEQ. ID. NO: 14) |
| Otx1(425): | GCT GTT CGC AAA GAC TCG CTA C | (SEQ. ID. NO: 15) |
| | ATG GCT CTG GCA CTG ATA CGG ATG | (SEQ. ID. NO: 16) |
| Otx2(211): | CCA TGA CCT ATA CTC AGG CTT CAG G | (SEQ. ID. NO: 17) |
| | GAA GCT CCA TAT CCC TGG GTG GAA AG | (SEQ. ID. NO: 18) |
| Pax2(545): | CCA AAG TGG TGG ACA AGA TTG CC | (SEQ. ID. NO: 19) |
| | GGG ATA GGA AGG ACG CTC AAA GAC | (SEQ. ID. NO: 20) |
| Pax 5(451): | CAG ATG TAG TCC GCC AAA GGA TAG | (SEQ. ID. NO: 21) |
| | ATG CCA CTG ATG GAG TAT GAG GAG CC | (SEQ. ID. NO: 22) |
| FGFR3(326): | ATC CTC GGG AGA TGA CGA AGA C | (SEQ. ID. NO: 23) |
| | GGA TGC TGC CAA ACT TGT TCT C | (SEQ. ID. NO: 24) |
| fgf8(312): | CAT GTG AGG GAC CAG AGC C | (SEQ. ID. NO: 25) |
| | GTA GTT GTT CTC CAG CAG GAT C | (SEQ. ID. NO: 26) |
| En1(381): | TCA AGA CTG ACT CAC AGC AAC CCC | (SEQ. ID. NO: 27) |
| | CTT TGT CCT GAA CCG TGG TGG TAG | (SEQ. ID. NO: 28) |
| Shh(354): | GGA AGA TCA CAA GAA ACT CCG AAC | (SEQ. ID. NO: 29) |
| | GGA TGC GAG CTT TGG ATT CAT AG | (SEQ. ID. NO: 30) |

Results

Although generally associated with CNS stem cells, both nestin and Otx2 signals were present in the undifferentiated ES cell cultures, probably the result of heterogeneity in the ES cell cultures wherein some cells expressing genes characteristic of CNS stem cells are present in the culture.

The SHH receptors smoothened (smo), patched (ptc) and the downstream mediator Gli1 were expressed during ES cell culture. Smo, ptc and Gli1 were also constitutively expressed in differentiating ES cells from early stages.

Expression of SHH and FGF8 as well receptor FGFR3 appeared only during stage 3 of ES cell culture.

The intermediate filament protein nestin and homeobox gene Otx2 were expressed before the Otx2 homologue Otx1 (FIG. 2). Expression of early paired-domain transcription factors Pax-2 and Pax-5 and the secreted factor Wnt1 precedes that of the homeodomain transcription factor engrailed 1 (En1). During the expansion stage, the ratio of Otx1:Otx2 shifts and En1 and FGF8 levels rise.

FIG. 2 demonstrates that ES cells progressively acquire mesencephalic cell fate during development. Numbers at the top of the panel designate stage of culture defined in FIG. 1.

Conclusion

The results indicate that at least some of the cells undergo a progressive commitment to CNS, mesencephalic and midbrain dopaminergic neuronal cell fates (FIG. 2). The gene expression of the in vitro ES was similar to gene expression in the developing CNS, in vivo.

Although no TH expression was detected at stages 1-4 of ES cell culture, gene expression data suggest that the cultures contain precursors that may be primed for induction of TH but require time and appropriate conditions to express differentiated neuronal features.

Overall, the results of this analysis demonstrate that our multi-step ES cell culture protocol (stages 1-4, FIG. 1) results in a progressive induction of a large number of genes that are expressed in the precursors for midbrain dopaminergic neurons.

### Example 11

### Synaptic Activity of Differentiated Cell Culture

In this example, the synaptic activity of the differentiated neurons in the cell culture was examined. Previous experiments (data not shown) revealed that the differentiated neurons released dopamine when depolarized. This example demonstrates that sustained trains of action potentials, characteristic of mature neurons, were observed when the cells were depolarized (n=54). The cells respond to neurotransmitters (GABA/glutamate) and showed spontaneous synaptic activity that was blocked by inhibitors of action potentials. Biocytin labeling was used to show that many recorded neurons were dopaminergic.

Briefly, ES cells were differentiated as described above. After 13 days of differentiation, the cells were transferred to a recording medium (described below). A patch electrode was used to record electrical activity of individual neurons within the cell culture (and, alternately, to stimulate individual cells within the cell culture). A multiple electrode system was used so that the electrical activity of up to 3 cells could be recorded at one time. The recordings show that the cells in the culture are spontaneously active. The synaptic activity of the cells was examined by the application of tetrodotoxin (TTX). The presence of receptors for GABA and glutamate was evaluated by application of of GABA and glutamate to the dendrites and monitoring the resulting electrical activity. Biocytin (a tracer) was used to label the cells that were monitored with the patch electrode. The cells were then fixed and evaluated for the presence of biocytin and TH to determine whether the synaptically active cells were dopaminergic cells.

More specifically, the differentiated cells were transferred to a recording medium containing 130 mM NaCl, 4 mM KCl, 2 mM CaCl₂, 1 mM MgCl₂, 10 mM HEPES, 10 mM glucose (pH 7.35, 325 mOsm). Patch pipettes were filled with 110 mM K-Gluconate, 20 mM KCl 2 mM Mg-ATP, 10 mM Na₂ phosphocreatine, 1.0 mM EGTA, 0.3 mM GTP-Tris, and 20 mM HEPES (pH 7.25, 320 mOsm). Biocytin (0.2%) was added to the intracellular medium daily. Recordings were performed under visual guidance using a Zeiss Axioskop microsope (Zeiss, Germany). Cells were recorded in voltage clamp, holding potential −60 to −70 mV. Signals were amplified using an Axopatch 200B amplifier and data was acquired and analyzed on a PC using pClamp 8 (Axon Instruments, USA). Current clamp recordings were performed with a second amplifier (AxoClamp 2B, Axon Instruments, USA). Neurotransmitters (glutamate and GABA, 10 mM) were applied by pressure application (Picospritzer, General Valve).

As shown in FIG. 9A, the differentiated dopaminergic neurons (13 days) in the cell culture fire an initial action potential followed by a few others at low frequency upon depolarization. When the amount of depolarization is increased, the neurons fire a train of action potentials at a higher frequency. This is characteristic of mature neurons.

As shown in FIG. 9B, the dopaminergic neuron an inward current is apparent upon application of GABA to the dendrites of the differentiated neuron.

As shown in FIG. 9C, glutamate application to the dendrites of a differentiated dopaminergic neuron also leads to an inward current.

As shown in FIG. 9D, the spontaneous activity in differentiated dopaminergic neurons (recorded in voltage clamp mode) diminished when action potentials were blocked with tetrodotoxin (TTX, 1 mm). TTX blocks almost all spontaneous activity indicating that most of the activity is due to synaptic release of transmitter, evoked by spontaneous action potential firing in presynaptic neurons.

### Example 12

### Generation of Midbrain Dopaminergic Neurons from Nurr1-Transduced Embryonic Stem Cells

Nurr1, an orphan nuclear receptor, is associated with the induction of midbrain dopaminergic neurons during neurogenesis and for the maintenance of dopaminergic phenotype during adulthood. In this Example, totipotent mouse embryonic stem cells (ES) cells were transduced with Nurr1. Transduction with Nurr1 increased differentiation of functional dopaminergic neurons.

A. Construction of Plasmid pCMV-Nurr1

Plasmid pCMV-Nurr1 was constructed using the pCVM-Script® Vector from Stratagene via known methods.

B. Construction of pcDNA3.1-Neomycin Plasmid

The pcDNA3.1 neomycin plasmid was constructed using the pMc1 -neo Vector from Stratagene via known methods.

C. Nurr1 Transfection

ES cells were cotransfected with a pCMV-Nurr1 and pcDNA3.1-neomycin resistance plasmid via electroporation. Standard conditions were employed as described by Brigid Hogan et al., (1994) Manipulating the Mouse Embryo: A Laboratory Manual, (Second Edition), Cold Spring Harbor Laboratory Press.

D. Selection of Nurr1-Transfected Clones

The Nurr1-transfected clones were selected using standard conditions described by Brigid Hogan et al., (1994) Manipulating the Mouse Embryo: A Laboratory Manual, (Second Edition), Cold Spring Harbor Laboratory Press. A total of 27 stable Nurr1-transfected clones were selected by growth in the presence of neomycin (200 μg/ml).

E. Nurr1 Expression

Overexpression of Nurr1 was determined using an antibody against a tag incorporated into the expressed protein via known methods. All 27 clones overexpressed Nurr1. The three clones with the highest expression were chosen for further analysis.

F. Clonal Expansion

Nurr1 positive cells were expanded using known methods to generate a culture of transfected undifferentiated cells.

G. In Vitro Differentiation

The Nurr1-transfected ES cells were differentiated, following the method described in Examples 1 through 5. Briefly, undifferentiated ES cells transfected with Nurr1 were maintained on gelatin-coated dishes in knockout ES medium containing 15% ES-qualified serum with supplements (stage I). For differentiation, ES cells were dissociated into single cells, and cultured on nonadherent petri-dishes for 4 days in the presence of leukemia inhibitory factor (1,400 units/ml) to generate embryoid bodies (stage II). The embryoid bodies were then plated on adhesive tissue culture plates for 24 hours and transferred to serum-free ITS medium containing fibronectin for 10 days to select for neural precursor cells (stage III). These cells were dissociated and grown in N2 medium for 4 days on coverslips which were coated with poly-L-ornithine (15/μg/ml)/fibronectin (1 μg/ml) in the presence of FGF2 (20 ng/ml), Shh (500 ng/ml) and FGF8 (100 ng/ml) (stage IV). FGF2 was removed to induce differentiations and ascorbic acid (200 mM) was added to the medium (stage V) and the cells were cultured for an additional 8-12 days before characterization.

H. Immunocytochemical Analysis

Immunohistochemical staining was performed essentially as described in Example 7 using the following antibodies: TH polyclonal 1:400 (Pel-Freeze), TH monoclonal 1:1000 (Sigma) α-tubulin type III (TuJ1) monoclonal 1:500 (Babco), serotonin polyclonal 1:4000 (Sigma), DAT monoclonal 1:5000 (Chemicon), En-1 monoclonal 1:50 (Developmental Studies Hybridoma Bank, and fluorescently labeled secondary antibodies (Jackson Immunoresearch Laboratories).

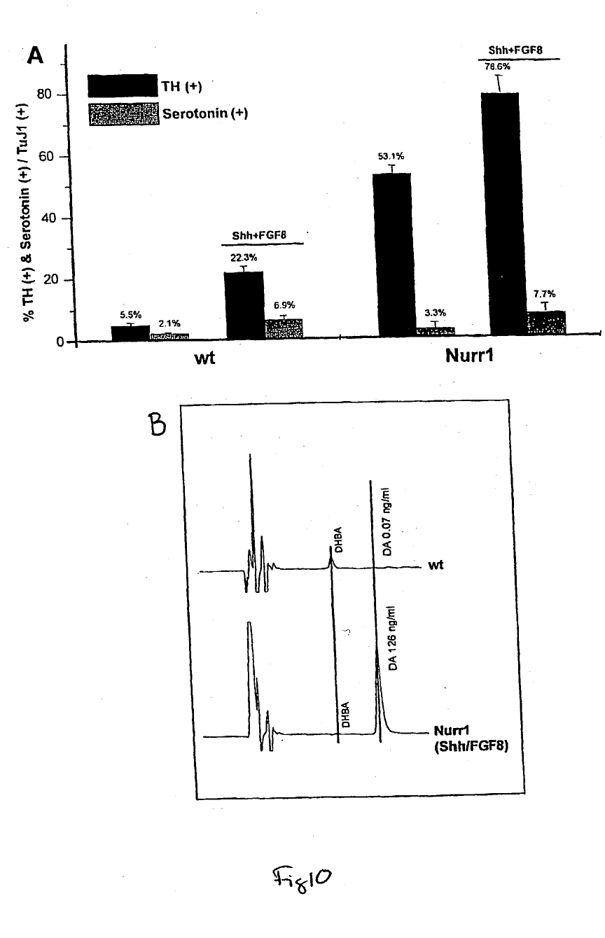

Of the total cells in the differentiated population, 8.25% were TuJ1+, a specific marker for neurons. As shown in FIG. 10A, in wild-type ES cells (non-transfected), 5.5% of TuJ1+ neurons were TH+. Nurr1 overexpression increased the TH+ population by 10-fold (53.1% of TuJ1 neurons were TH+). At the same time, the proportion of serotonin+ neurons was not significantly changed. Shh and FGF8 have been previously shown to promote ventral midbrain fates in neural plate explant and wild-type ES cells. In our culture system, addition of Shh/FGF8 during stage IV increased the TH+ population from both wild-type and Nurr1 transfected ES cells (22.3% of TuJ1 in wild-type; 78.6% of TuJ1+ in Nurr1-transfected ES cells). Treatment with Shh/FGF8 in other stages had no effect on population of TH+ cells.

I. Characterization of TH+ Cells as Ventral Mesencephalic Dopaminergic Neurons

To confirm the phenotype of the differentiated TH+neurons, three definitive characteristics for ventral meseneephalic dopaminergic neurons were examined.

i. Eigrailed (En-1)

The hoemobox transcription factor, Engrailed (En-1) has been known as a mediator of midbrain dopaminergic development. Gene expression continues into adulthood in substantia nigra. The differentiated neurons were examined for the presence of En-1 using standard procedures for immunocytochemistry.

Most (i.e., more than 70%) of the TH+ neurons derived from Nurr1-transfected ES cells expressed En-1, typically in the nucleus, and the expression was continued at later stages in postmitotic differentiating TH+ cells (Data not shown).

ii. Dopamine Transporter (DAT)

Dopamine Transporter (DAT) is another marker of substantia nigra dopaminergic neurons and know to be a regulator of dopamine neurotransmission. The differentiated neurons were examined for the presence of DAT using standard procedures for immunocytochemistry. The culture included about 20% DAT-immunoreactive neurons after long term maturation of Nurr1-transfected ES cells in serum-free N2 medium (FIG. 10C).

iii. Dopamine Release

To further confirm the generation of dopaminergic neurons, the release of dopamine in Nurr1 ES cells was measured using reverse phase HPLC as described by Studer et al., “Transplantation of expanded mesencephalic precursor leads to behavioral recovery in Hemiparkinsonian rats,” Nature Neurosci. 1:290-295 (1998). Briefly, the differentiated cells were incubated in HBSS (56 mM KCl) and supernatants were stabilized with orthophosphoric acid (7.5%)/metabisulfate (0.22 mg/ml), and stored at −80° C. until analysis.

In response to depolarization, neurons from Nurr1-transfected cells released 1000 fold more dopamine than wild-type ES cell derived neurons (FIG. 10B). The wild type cells released only 0.07 ng/ml dopamine upon stimulation. In contrast, the Nurr1-transfected cells released 128 ng/ml.

J. Behavioral Studies

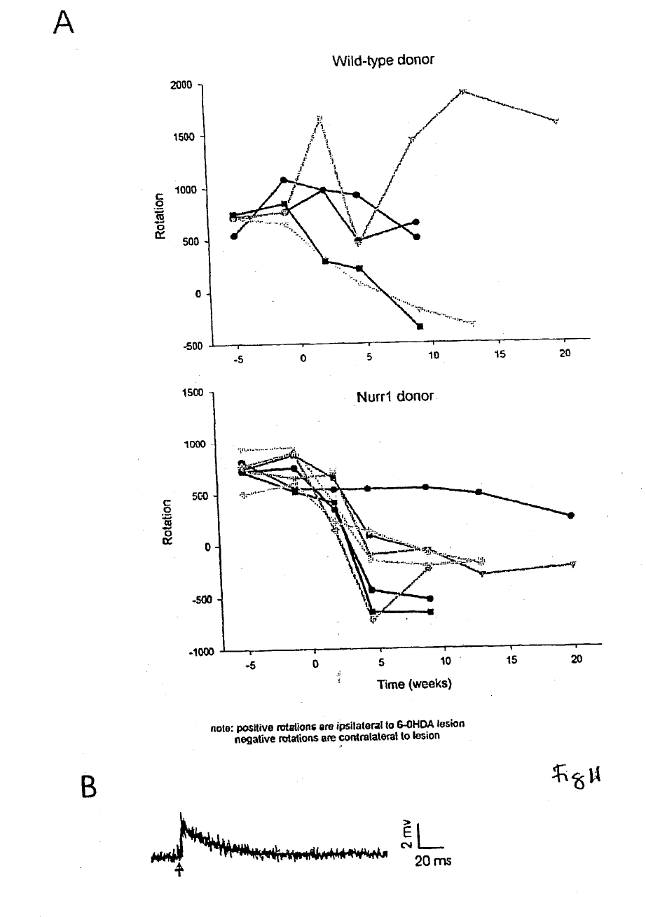

Rats were lesioned and grafted with differentiated wild-type and Nurr1-transfected cells as described by Studer et al., “Transplantation of expanded mesencephalic precursor leads to behavioral recovery in Hemiparkinsonian rats,” Nature Neurosci. 1:290-295 (1998). As shown in FIG. 11A, 2 of 5 animals showed rotational correction when grafted with differentiated wild-type cells. As shown in FIG. 11B, 8 of 9 animals showed rotational correction when grafted with differentiated Nurr1-transfected cells. Zero (0) rotation is normal.

K. Electrophysiological Experiments

Following behavioral testing, electrophysiological experiments were performed on brain slices prepared from the striatum (including the graft site) of the grafted animals.

Patch clamp recordings revealed that the grafted cells formed synaptically functional TH+ neurons. Recording from a host striatal neuron while simulating cells in the graft demonstrates that the grafted cells form synapses with the host neurons (FIG. 11B).

All references cited herein are hereby incorporated by reference in their entirety.

1. A method of culturing cells to produce a population of cells comprising neuronal cells, wherein the method comprises:
a. expanding undifferentiated embryonic stem cells in the presence of Leukemia Inhibitory Factor (LIF) and dissociating the undifferentiated embryonic stem cells to form a population comprising a majority of single cells;
b. generating embryoid bodies from the population comprising the majority of single cells;
c. culturing the embryoid bodies to select for central nervous system precursor cells;
d. expanding the central nervous system precursor cells by culturing the central nervous system precursor cells in an expansion medium that comprises at least one neurologic factor and that lacks 4-(2-hydroxyethyl)-1-piperazine-ethanesulfonic acid;
e. differentiating the expanded central nervous system precursor cells to form a culture of differentiated neuronal cells by culturing the expanded central nervous system precursors a culture medium that lacks the neurologic factor,
thereby producing the population of cells comprising ventral neuronal cells.

2. The method of claim 1, wherein the neurologic factor is selected from the group consisting of bFGF, SHH, FGF8, combinations thereof and functional fragments thereof

3. The method of claim 1, wherein the step of differentiating the expanded central nervous system precursor cells to form differentiated neuronal cells comprises culturing the expanded nervous system precursors in a culture medium that lacks bFGF.

4. The method of claim 1, wherein the population of ventral neuronal cells comprises at least about 30% dopaminergic neurons.

5. The method of claim 1, wherein the step of expanding a culture of embryonic stem cells comprises culturing embryonic stem cells for about 4 to about 7 days.

6. The method of claim 1, wherein the embryonic stem cells are human embryonic stem cells.

7. The method of claim 1, wherein the step of expanding a cell culture of embryonic stem cells comprises culturing embryonic stem cells on tissue culture plates.

8. The method of claim 1, wherein the step of expanding a cell culture of embryonic stem cells comprises culturing embryonic stem cells on gelatin coated tissue culture plates.

9. The method of claim 1, wherein the step of generating embryoid bodies comprises culturing expanded embryonic stem cells for about 4 to about 7 days.

10. The method of claim 1, wherein the step of generating embryoid bodies comprises culturing expanded undifferentiated embryonic stem cells in suspension.

11. The method of claim 1, wherein the step of culturing the embryoid bodies to select for central nervous system precursor cells comprises culturing the embryoid bodies in a serum-free medium.

12. The method of claim 1, wherein the step of culturing the embryoid bodies to select for central nervous system precursor cells comprises culturing the embryoid bodies on a fibronectin-coated surface.

13. The method of claim 1, wherein the step of culturing the embryoid bodies to select for central nervous system precursor cells comprises culturing the embryoid bodies for about 6 to about 8 days.

14. The method of claim 1, wherein the step of differentiating the expanded central nervous system precursor cells comprises culturing the expanded central nervous system precursors in a medium which comprises ascorbic acid.

15. The method of claim 1, wherein the population of differentiated neuronal cells comprises dopaminergic neurons.

16. The method of claim 1, wherein the differentiated neuronal cells comprise at least about 30% dopaminergic neurons.

17. The method of claim 1, wherein the differentiated neuronal cells comprise at least about 10% serotonergic cells.

18. The method of claim 1, further comprising a step of transfecting the undifferentiated embryonic stem cells with a gene encoding Nurr1.

19. A method of culturing dopaminergic neuronal cells, comprising:
a. generating embryoid bodies from the suspension of single embryonic stem cells;
b. selecting central nervous system precursor cells;
c. expanding the central nervous system precursor cells by culturing the central nervous system precursor cells in an expansion medium that comprises at least one neurologic agent and lacks 4-(2-hydroxyethyl)-1-piperazine-ethanesulfonic acid; and
d. differentiating the expanded nervous system precursor cells in an expansion medium that lacks the neurologic agent to form a culture of differentiated neuronal cells that comprises at least about 30% dopaminergic neurons.

20. The method of claim 19, wherein the neurologic agent is selected from the group consisting of basic fibroblast growth factor, sonic hedgehog protein, fibroblast growth factor-8, functional fragments thereof and combinations thereof.

21. A method of treating a patient with neurological disorder, comprising the steps of:
administering a culture of differentiated neuronal cells to the patient wherein the culture of differentiated neuronal cells comprises at least about 30% dopaminergic neurons.

22. The method according to claim 21 wherein the differentiated neuronal cell culture is derived from embryonic stem cells.

23. The method according to claim 22 wherein the differentiated neuronal cell culture is derived from human embryonic stem cells.

24. The method according to claim 22 wherein the differentiated neuronal cell culture is derived from murine embryonic stem cells.

25. The method according to claim 21, wherein the culture of differentiated neuronal cells is prepared by a method comprising:
a. expanding undifferentiated embryonic stem cells in the presence of Leukemia Inhibitory Factor (LIF) and dissociating the undifferentiated embryonic stem cells to form a population comprising a majority of single cells;
b. generating embryoid bodies from the population comprising the majority of single cells;
c. culturing the embryoid bodies to select for central nervous system precursor cells;
d. expanding the central nervous system precursor cells by culturing the central nervous system precursor cells in an expansion medium that comprises at least one neurologic factor and that lacks 4-(2-hydroxyethyl)-1-piperazine-ethanesulfonic acid;
e. differentiating the expanded central nervous system precursor cells to form a culture of differentiated neuronal cells by culturing the expanded central nervous system precursors in a culture medium that lacks the neurologic factor.

26. The method of claim 25, wherein the method of preparing the culture of differentiated neuronal cells further comprises a step of transfecting the undifferentiated embryonic stem cells with a gene encoding Nurr1.

27. The method of claim 21, wherein the neurological disorder is Parkinson's disease.

28. A method of introducing a gene product into a brain of a patient, comprising:
a. transfecting embryonic stem cells;
b. culturing the transfected embryonic stem cells to provide a culture of differentiated neuronal cells comprising at least about 30% dopaminergic neurons; and
c. administering said differentiated transformed neuronal cells into a patient in need thereof.

29. The method of claim 28, wherein the culture of differentiated neuronal cells is generated by a method comprising:
a. expanding undifferentiated embryonic stem cells in the presence of Leukemia Inhibitory Factor (LIF) and dissociating the undifferentiated embryonic stem cells to form a population comprising a majority of single cells;
b. generating embryoid bodies from the population comprising the majority of single cells;
c. culturing the embryoid bodies to select for central nervous system precursor cells;
d. expanding the central nervous system precursor cells by culturing the central nervous system precursor cells in an expansion medium that comprises at least one neurologic factor and that lacks 4-(2-hydroxyethyl)-1-piperazine-ethanesulfonic acid;
e. differentiating the expanded central nervous system precursor cells to form a culture of differentiated neuronal cells by culturing the expanded central nervous system precursors in a culture medium that lacks the neurologic factor.

30. The method of claim 29, wherein said transfected cell produces a gene product selected from the group consisting of tyrosine hydroxylase, nerve growth factor (NGF), brain derived neurotrophic factor (BDNF), basic fibroblast growth factor (bFGF), glial derived growth factor (GDNF) NT-3, and NT-4/5.

31. An assay for a substance, comprising:
a. providing a culture of differentiated neuronal cells comprising at least 30% dopaminergic neurons;
b. exposing said differentiated neuronal cells to the substance; and
c. observing the effect of the substance on the differentiated neuronal cells.

32. The method of claim 3 1, wherein the culture of differentiated neuronal cells is generated by a method comprising:
a. expanding undifferentiated embryonic stem cells in the presence of Leukemia Inhibitory Factor (LIF) and dissociating the undifferentiated embryonic stem cells to form a population comprising a majority of single cells;
b. generating embryoid bodies from the population comprising the majority of single cells;
c. culturing the embryoid bodies to select for central nervous system precursor cells;
d. expanding the central nervous system precursor cells by culturing the central nervous system precursor cells in an expansion medium that comprises at least one neurologic factor and that lacks 4-(2-hydroxyethyl)-1-piperazine-ethanesulfonic acid;
e. differentiating the expanded central nervous system precursor cells to form a culture of differentiated neuronal cells by culturing the expanded central nervous system precursors in a culture medium that lacks the neurologic factor.

33. The method of claim 32, wherein the step of differentiating the expanded central nervous system precursor cells to form differentiated neuronal cells comprises culturing the expanded central nervous system precursors in a medium which includes ascorbic acid.

34. A cell culture comprising about 50% to about 85% neurons which comprise between about 20% and 40% dopaminergic neurons and between about 1% to about 3% astrocytes.

35. The cell culture of claim 34 wherein the differentiated neuronal cells comprise dopaminergic cells that are functional in vivo.

36. The cell culture of claim 34 wherein at least some of the differentiated neuronal cells are synaptically active.

37. The cell culture of claim 34 wherein the differentiated neuronal cells comprise an exogenous Nurr1 gene.

38. A method of culturing neurons from embryonic stem cells, comprising:
a. expanding undifferentiated the embryonic stem cells on a surface that inhibits differentiation cells in the presence of Leukemia Inhibitory Factor (LIF);
b. disengaging the embryonic stem cells from the surface in clusters;
c. dissociating the clusters of embryonic stem cells to obtain a population which includes a majority of individual cells;
d. generating embryoid bodies in suspension;
e. culturing the embryoid bodies on a coated surface in serum free medium to select for Central Nervous System (CNS) precursor cells;
f. expanding the CNS precursor cells by culturing the cells in an expansion medium that comprises at least one neurologic agent selected from SHH, FGF8, EFG and bFGF; and
g. differentiating the expanded CNS precursor cells to form neurons by withdrawing the at least one neurologic agent from the culture.

39. The method according to claim 38, further comprising adding a differentiation enhancing agent to the culture of central nervous system precursor cells.

40. The method according to claim 39, wherein the differentiation enhancing agent comprises ascorbic acid.

41. The method according to claim 38, wherein the step of culturing the embryoid bodies to select for CNS precursor cells comprises culturing the embryoid bodies in the presence of one or more of differentiation enhancing agents, wherein the differentiation enhancing agent is selenium, insulin, transferrin, or fibronectin.

42. The method of claim 38, further comprises a step of transfecting the undifferentiated embryonic stem cells with a gene encoding Nurr1 prior to the step of expanding the culture of undifferentiated embryonic stem cells.

43. The method of claim 38, wherein the step of expanding the CNS precursor cells comprises culturing the CNS precursor cells in a 4-(2-hydroxyethyl)-1-piperazine-ethanesulfonic acid (HEPES) free medium.

44. The method of claim 1, wherein the culture medium is N2 medium.

45. The method of claim 19, wherein the culture medium is N2 medium.

46. The method of claim 1, wherein the expansion media comprises fibroblast growth factor-8 and sonic hedgehog protein.

46. The method of claim 19, wherein the expansion media comprises fibroblast growth factor-8 and sonic hedgehog protein.
